(19) United States
(12) Patent Application Publication
(10) Pub. No.: US 2007/0065394 A1
(43) Pub. Date: Mar. 22, 2007

(54) Compositions effective in altering the perception of malodor

(76) Inventor: Virginia Ruth Pinney, The Woodlands, TX (US)

Correspondence Address: Martin D Moynihan;PRTSI Inc, P O Box 16446, Arlington, VA, 22215, US

(21) Appl. No.: 10/579,842

(22) PCT Filed: Nov. 15, 2004
(86) PCT No.: PCT/IL04/01047

§ 371 Date: May 17, 2006

Related U.S. Application Data
(60) Provisional application No. 60/520,319, filed on Nov. 17, 2003.
(60) Provisional application No. 60/526,600, filed on Dec. 4, 2003.

(51) Int. Cl. A61K 8/97; A61K 36/73; A61K 36/67; A61K 36/324; A61K 36/32

(52) U.S. Cl. 424/74; 424/725; 424/734; 424/765

(57) ABSTRACT

A method of modifying perception of a malodor, such as the stench associated with putrefying flesh, by selectively affecting specific olfactory receptors in an individual is disclosed. Disclosed are articles of manufacture and especially innovative compositions effective in implementing the method of the present invention including at least three plant extracts.

### FIELD AND BACKGROUND OF THE INVENTION

The present invention relates to the field of scents, and particularly to a composition that alters the perception of odors especially of malodors such as those produced by putrefying flesh and the like.

The sense of smell is perhaps the most primitive of the five senses and, as such, is of critical importance to the survival of an organism. Scents detected by the sense of smell can inform the organism whether a given environment is attractive or repulsive. Attractive environments identified by smell include potential mates and edible foods. Repulsive environments identified by smell include harmful items resembling food, danger sensed by a member of the cohort, volatile poisons, excrement, rot and death. When an organism encounters a smell indicative of a repulsive environment, the organism feels an urge to leave the vicinity of the environment. An organism that cannot leave a repulsive area suffers varying degrees of discomfort. In higher organisms, including humans, such discomfort can include various levels of incapacitation, vomiting, loss of consciousness and a long lasting memory of the odor that is associated with the emotions of the situation at the time of the perception.

The association of scents with emotions and autobiographical experiences is generally referred to as the Proust phenomenon (see Chu and Downes Chem. Senses 2000, 25, 111-116) Through a not entirely understood mechanism, signals generated by stimulation of certain olfactory receptors or certain combinations of olfactory receptors are directed not only to parts of the brain that analyze and identify the smell and its source, but produce an involuntary physical or emotional effect. Such effects commonly include uncontrollable anxiety, gagging, nausea, nervousness, disgust or revulsion when associated with unpleasant smells or smells associated with unpleasant experiences. In some cases, smells trigger a type of post-traumatic stress disorder when coupled to an exceptionally unpleasant past experience. Most commonly, military, rescue and medical personnel suffer from such effects, especially from smells associated with exceptionally gruesome and horrible deaths. It is also known that certain types of unpleasant medical treatments include anticipatory nausea. For example, patients afflicted with cancer undergoing chemotherapeutic treatment often feel ill just by smelling the hospital or clinic where treatment takes place (Boakes et al. Eur. J. Cancer 1993, 29A(6), 866-870).

It has known that malodors may reduce the efficacy with which a person performs a complex task (Danuser et al. Human Factors 2003, 45(4), 549-562 and references therein). However, the realities of modern society often necessitate that humans perform highly sensitive and accurate tasks in the presence of the most foul malodors and stenches. Such tasks include: medical treatment, especially in the fields of gastrointestinal surgery, emergency medical procedures, treatment of burn and gangrenous wounds, and periodontal surgery; rescue operations, especially resulting from earthquakes or massive accidents; forensic operations, including pathology laboratories; grave excavations; and work related to abattoirs.

A number of methods have been developed for ameliorating the effects of malodors. A general background of various innovative methods in the field of overcoming malodors may be found in U.S. Pat. No. 3,091,511, U.S. Pat. No. 3,923,005, U.S. Pat. No. 4,909,986, U.S. Pat. No. 4,959,207, U.S. Pat. No. 4,989,727, U.S. Pat. No. 5,416,116, U.S. Pat. No. 5,500,138, U.S. Pat. No. 5,534,165, U.S. Pat. No. 5,531,910, U.S. Pat. No. 5,539,034, U.S. Pat. No. 5,668,097, U.S. Pat. No. 5,714,137, U.S. Pat. No. 5,718,887, U.S. Pat. No. 5,780,020, U.S. Pat. No. 5,783,544, U.S. Pat. No. 5,942,217, U.S. Pat. No. 5,968,404, U.S. Pat. No. 6,495,097, the PCT patent application published as WO93/07853 and references cited therein. The methods described therein generally involve the use of chemical compounds and compositions either to mask malodors or to neutralize malodors at the source.

Neutralizing odors at the source generally involves eliminating microorganisms responsible for the odor (e.g., using antibacterial agents and antifungal agents such as sodium hypochlorite), neutralizing odor causing molecules or adsorbing odor-causing molecules (e.g., onto talc or activated carbon). However, neutralizing odors is often not practical. The size of the source to be neutralized may be too large, the malodor may be intrinsic to the source, e.g. sewage processing or neutralization may damage the source, e.g. medical and forensic applications.

Masking malodors is well known in the art, for instance by dispensing vapors of an inoffensive or sweet-smelling composition (e.g., perfumes and fragrances) in a volume so as to overload the sense of smell. The volume can be large such as a room or small, for example in the vicinity of the nose of an individual.

Masking malodors is not always effective especially when the malodors to be masked are intense. In some instances, chemicals used to mask odors are toxic, inflammable, intoxicating or otherwise hazardous. Masking certain exceptionally offensive odors is ineffective using known compositions, such as the malodor associated with the rotting or putrefaction of flesh. Even if effective, masking makes it difficult or impossible to detect other smells that may be important, for example detecting gas leaks.

There is a widely recognized need for, and it would be highly advantageous to have a method of ameliorating at least some of the negative effects of malodor and stench such as that of putrefying or rotting flesh not having the disadvantages of the methods known in the art. It would be advantageous to have a method of neutralizing the coupling of certain smells with undesired emotions or debilitating physical effects.

### SUMMARY OF THE INVENTION

The present invention provides a method, a composition and an article of manufacture all for ameliorating at least some of the negative effects of malodors on an individual.

According to the teachings of the present invention there is provided a method of modifying perception of a malodor comprising selectively affecting specific olfactory receptors in an individual thereby altering perception of the malodor. According to one aspect of the present invention the alteration of perception is achieved by exposing the individual to a composition capable of binding to the appropriate olfactory receptors. According to a feature of the present invention such a composition includes volatile components. In a preferred embodiment of the present invention, such a composition includes at least three plant extracts, for example a composition of the present invention.

According to the teachings of the present invention there is also provided a composition effective in altering the perception of at least one malodor by affecting at least one olfactory receptor. In an embodiment of the present invention, the composition comprising at least three plant extracts, at least four plant extracts or even at least five plant extracts.

According to a feature of the present invention the three, four or five plant extracts are selected from the group consisting of frankincense, extract of roses, extract of Cananga, extract of Piper, and extract of Bursera.

In an embodiment of the present invention, the frankincense (preferably from Boswellia carteri) comprises between about 0.1% and about 40% by weight of the composition.

In an embodiment of the present invention, the extract of roses comprises an extract of rose flowers. In an embodiment of the present invention, the extract of rose flowers comprises an extract of rose petals, especially petals of Rosa damascena, for example of roses grown in Bulgaria. In embodiments of the present invention, the extract of rose petals is rose oil, rose oil absolute or a mixture thereof. In an embodiment of the present invention, the extract of roses comprises between about 0.1% and about 40% by weight of the composition.

In an embodiment of the present invention, the extract of Cananga is an extract of Cananga odorata, especially the flower of Cananga, especially the essential oil of the flower of Cananga. In an embodiment of the present invention, the extract of Cananga is ylang ylang. In an embodiment of the present invention, the extract of Cananga comprises between about 0.1% and about 40% by weight of the composition.

In an embodiment of the present invention, the extract of Piper comprises an extract of leaves of a plant of Piper. In an embodiment of the present invention, the extract of Piper comprises an extract of Piper auritum preferably an extract of leaves of Piper auritum. In an embodiment of the present invention, the extract of Piper comprises an essential oil of leaves of Piper auritum. In an embodiment of the present invention, the extract of Piper comprises between about 0.1% and about 60% by weight of the composition.

In an embodiment of the present invention, the extract of Bursera comprises an extract of Bursera wood. In an embodiment of the present invention, the extract of Bursera wood comprises linaloe wood oil. In an embodiment of the present invention, the extract of Bursera wood comprises oil extracted from Bursera glabrifolia and/or Bursera delpechiana. In an embodiment of the present invention, the extract of Bursera comprises between about 0.1% and about 22% by weight of the composition.

In an embodiment of the present invention, the composition further comprises at least one auxiliary component selected from the group consisting of vanillin, ethyl vanillin, extract of Hedychium, extract of Menta, extract of Citrus and mixtures thereof. In an embodiment of the present invention, the auxiliary component comprises up to about 60% by weight of the composition.

In an embodiment of the present invention including extract of Hedychium, the extract is preferably Hedychium spicatum, preferably an extract of the root of Hedychium, preferably an essential oil of the root of Hedychium, especially the essential oil of the root of Hedychium spicatum.

In an embodiment of the present invention including extract of Menta, the extract is preferably an extract of Menta piperata, preferably an oil of Menta, preferably an essential oil of Menta, especially the essential oil of Menta piperata.

In an embodiment of the present invention including an extract of Citrus, the extract is preferably an extract of Citrus limonum, preferably an oil of Citrus, preferably an essential oil of Citrus, especially an essential oil of Citrus limonum.

In an embodiment of the present invention, the auxiliary component comprises vanillin and at least one additional auxiliary component selected from the group consisting of ethyl vanillin, extract of Hedychium, extract of Menta, extract of Citrus. In such an embodiment, the vanillin preferably comprises up to about 58% by weight of the composition.

In an embodiment of the present invention, the auxiliary component comprises ethyl vanillin and at least one additional auxiliary component selected from the group consisting of vanillin, extract of Hedychium, extract of Menta, extract of Citrus. In such an embodiment, the ethyl vanillin preferably comprises up to about 58% by weight of the composition.

In an embodiment of the present invention, the composition further comprises benzyl benzoate. In an embodiment of the present invention, the benzyl benzoate comprises between about 0.1% and about 60% by weight of the composition.

In an embodiment of the present invention, the composition further comprises an aldehyde component. In an embodiment of the present invention, the aldehyde component comprises a mixture of one or more hydrocarbon aldehydes of a structure RCH═O, wherein R is selected from the group consisting of aryl and alkyl groups, preferably R being alkyl groups having between 8 and 16 carbon atoms. In an embodiment of the present invention, the aldehyde component comprises a mixture of at least two alkyl aldehydes selected from the group consisting of aldehyde C8, aldehyde C9, aldehyde C10, aldehyde C11, aldehyde C12, aldehyde C13/C15, aldehyde C14, aldehyde C16. In an embodiment of the present invention, the aldehyde component comprises between about 0.1% and about 55% of the composition.

In an embodiment of the present invention, the composition further comprises amyl salicylate. In an embodiment of the present invention, the amyl salicylate comprises iso-amyl salicylate, n-amyl salicylate or a mixture thereof. In an embodiment of the present invention, the amyl salicylate comprises between about 0.1% and about 55% by weight of the composition.

According to the teachings of the present invention there is provided a specific preferred composition suitable for implementing the method of the present invention, the composition comprising benzyl benzoate, an aldehyde component, amyl salicylate, frankincense, extract of roses, extract of Cananga, extract of Piper, extract of Bursera and at least one auxiliary component selected from the group consisting of vanillin, ethyl vanillin, extract of Hedychium, extract of Menta, extract of Citrus and mixtures thereof.

In an embodiment of the present invention, the benzyl benzoate comprises between about 0.1% and about 60% by weight of the composition.

In an embodiment of the present invention, the aldehyde component comprises a mixture of one or more hydrocarbon aldehydes of a structure RCH═O, wherein R is selected from the group consisting of aryl and alkyl groups, preferably R being alkyl groups having between 8 and 16 carbon atoms. In an embodiment of the present invention, the aldehyde component comprises a mixture of at least two alkyl aldehydes selected from the group consisting of aldehyde C8, aldehyde C9, aldehyde C10, aldehyde C11, aldehyde C12, aldehyde C13/C15, aldehyde C14, aldehyde C16. In an embodiment of the present invention, the aldehyde component comprises between about 0.1% and about 55% of the composition.

In an embodiment of the present invention, the amyl salicylate comprises iso-amyl salicylate, n-amyl salicylate or a mixture thereof. In an embodiment of the present invention, the amyl salicylate comprises between about 0.1% and about 55% by weight of the composition.

In an embodiment of the present invention, the frankincense preferably from Boswellia carteri) comprises between about 0.1% and about 40% by weight of the composition.

In an embodiment of the present invention, the extract of roses comprises an extract of rose flowers. In an embodiment of the present invention, the extract of rose flowers comprises an extract of rose petals, especially petals of Rosa damascena, for example of roses grown in Bulgaria. In embodiments of the present invention, the extract of rose petals is rose oil, rose oil absolute or a mixture thereof. In an embodiment of the present invention, the extract of roses comprises between about 0.1% and about 40% by weight of the composition.

In an embodiment of the present invention, the extract of Cananga is an extract of Cananga odorata, especially the flower of Cananga, especially the essential oil of the flower of Cananga. In an embodiment of the present invention, the extract of Cananga is ylang ylang. In an embodiment of the present invention, the extract of Cananga comprises between about 0.1% and about 40% by weight of the composition.

In an embodiment of the present invention, the extract of Piper comprises an extract of leaves of a plant of Piper. In an embodiment of the present invention, the extract of Piper comprises an extract of Piper auritum preferably an extract of leaves of Piper auritum. In an embodiment of the present invention, the extract of Piper comprises an essential oil of leaves of Piper auritum. In an embodiment of the present invention, the extract of Piper comprises between about 0.1% and about 60% by weight of the composition.

In an embodiment of the present invention, the extract of Bursera comprises an extract of Bursera wood. In an embodiment of the present invention, the extract of Bursera wood comprises linaloe wood oil. In an embodiment of the present invention, the extract of Bursera wood comprises oil extracted from Bursera glabrifolia and/or Bursera delpechiana. In an embodiment of the present invention, the extract of Bursera comprises between about 0.1% and about 22% by weight of the composition.

In an embodiment of the present invention including extract of Hedychium, the extract is preferably Hedychium spicatum, preferably an extract of the root of Hedychium, preferably an essential oil of the root of Hedychium, especially the essential oil of the root of Hedychium spicatum.

In an embodiment of the present invention including extract of Menta, the extract is preferably an extract of Menta piperata, preferably an oil of Menta, preferably an essential oil of Menta, especially the essential oil of Menta piperata.

In an embodiment of the present invention including an extract of Citrus, the extract is preferably an extract of Citrus limonum, preferably an oil of Citrus, preferably an essential oil of Citrus, especially an essential oil of Citrus limonum.

In an embodiment of the present invention, the auxiliary component comprises vanillin and at least one additional auxiliary component selected from the group consisting of ethyl vanillin, extract of Hedychium, extract of Menta, extract of Citrus. In such an embodiment, the vanillin preferably comprises up to about 58% by weight of the composition.

In an embodiment of the present invention, the auxiliary component comprises ethyl vanillin and at least one additional auxiliary component selected from the group consisting of vanillin, extract of Hedychium, extract of Menta, extract of Citrus. In such an embodiment, the ethyl vanillin preferably comprises up to about 58% by weight of the composition.

According to the teachings of the present invention there is also provided a method of preparing a composition of the present invention by combining the various components. For example when a preferred composition of the present invention is prepared, benzyl benzoate, frankincense, benzyl benzoate, frankincense, extract of roses, extract of Cananga, extract of Piper, and extract of Bursera are combined. According to a feature of the present invention, either or both of an aldehyde component and amyl salicylate are also combined with the other components of the composition. According to a feature of the present invention, at least one component selected from the group consisting of vanillin, ethyl vanillin, extract of Hedychium, extract of Menta, extract of Citrus is also combined with the other components of the composition. Preferably the frankincense is dissolved in the benzyl benzoate prior to mixing with the other ingredients.

According to the teachings of the present invention there is also provided an article of manufacture comprising a composition of the present invention (as described above) and a carrier. According to a feature of the present invention the article of manufacture is packaged in a packaging material and identified for use in ameliorating effects of malodor.

According to a feature of the present invention the carrier is selected from the group of fabric-care products, personal hygiene products, air-freshening products, cleaning products and cosmetic products.

Examples of fabric-care products useable as carriers for a composition of the present invention include but are not limited to laundry detergents, laundry soaps, fabric softeners, fabric sprays, fabric deodorants, dryer-added products, whitener products, bleaching products, optical whitener products and odor masking products.

Examples of cleaning products useable as carriers for a composition of the present invention include but are not limited to bleach, cleaners, dishwashing products, toilet cleaning products and floor cleaning products.

Examples of personal-hygiene products useable as carriers for a composition of the present invention include but are not limited to shaving creams, shaving lotions, after shave lotions, soaps, shampoos, hair conditioners, deodorants, sun-screen products, bath salts and bath oils.

Examples of cosmetic products useable as carriers for a composition of the present invention include but are not limited to perfumes, colognes, blushes, creams, face powders, lip balms and lip sticks.

According to a feature of the present invention the carrier is a liquid, a viscous fluid or a solid.

Examples of liquids suitable for use as carriers of the composition of the present invention include but are not limited to solutions, tinctures, oils, colognes, perfumes, eaux de parfum and eaux de toilette.

Examples of viscous fluids suitable for use as carriers of the composition of the present invention include but are not limited to balms, colloids, creams, emulsions, foams, gels, lotions, pastes, sols, smearable sticks, suspensions, and unguents. Exceptional suitable carriers include such smearable carriers as petrolatum, fractionated coconut oil, bees' wax and combinations thereof.

According to a preferred feature of the present invention, when a smearable carrier is petrolatum, fractionated coconut oil, bees' wax and the like, the carrier comprises above about 80%, generally between about 85% and about 99.99% (or even between about 85% and about 99.9% or even between about 85% and about 97%) by weight and the composition of the present invention preferably comprises between about 0.01% and about 15% (or between about 0.1% and about 15% or even between about 3% and about 15%) by weight of the article of manufacture.

Examples of solids suitable for use as carriers of the composition of the present invention include but are not limited to decorative items (e.g., artificial flowers), sublimating air fresheners, talc-based powders, carbon-based powders, fabrics, cloths, tissues, sponges, papers, pledgets, pads, nasal tampons, masks and bath salts.

According to the teachings of the present invention there is also provided a method of reducing the negative effects of malodor on an individual comprising positioning a composition (or an article of manufacture) of the present invention (especially a preferred composition as described hereinabove) so that vapors emanating from the composition affect olfactory receptors of the individual thereby reducing the negative effects on the individual of malodor emerging from a source of malodors found at or in a location.

Typical individuals for whom the method of the present invention are exceptionally applicable include but are not limited to pregnant women (who are sensitive to certain smells), wounded persons, persons afflicted with cancer, persons afflicted with AIDS, persons undergoing medical treatment, persons afflicted with a foul smelling wounds, diabetics, military personnel, health-care personnel (e.g., doctors, dentist, nurses, medics. paramedics) and rescue personnel (e.g. firemen, policemen).

Typical sources of malodor include but are not limited to agriculture, amines, body odor, cigarette smoke, compost, dairy industry, gangrenous wounds, garbage, human feet, diapers, dirty laundry, halitosis, bad breath, lesions, livestock, manure, mercaptans, sewage, sludge, smoke, swine, tobacco, trash compactors, tumors, smoke, stale sweat, ulcers, unwashed humans, vomit, waste water, rotting proteins (flesh), decomposing proteins (flesh), burned proteins (flesh), abscesses, urine, viscera, offal, feces, ammonia, amines produced during the putrefaction of proteins (flesh) and indoles produced during the putrefaction of proteins (flesh) and combinations thereof.

According to a further feature of the present invention the source is found in or at a location such as a clinic, dental clinic, hospital, surgical ward, mass grave, morgue, battlefields, abattoir, industrial plant, sewer, earthquake locus, collapsed building, sewage processing plant, tanning plant, animal handling area, barn, cancer ward, changing room, clarifier, coal mine, composting site, crematorium, crummy motel, diaper pail, dormitory, feed lot, garbage dump, garbage processing plant, kennel, landfill, laundry room, leather processing plant, locker room, lumber mill, meat processing plant, milking parlor, mine, mother in law, nursing home, old age home, outhouse, paper mill, photographic products manufacturing plant, poultry processing plant, prison, rendering plant, settling basin, sewage dewatering system, sludge station and sport center.

In an embodiment of the present invention, the positioned composition includes vanillin, ethyl vanillin or a combination thereof. Such a composition is exceptionally suited for use in reducing the negative effects of malodor found in locations including but not limited to hospitals, surgical wards, mass graves, morgues, battlefields, abattoirs, earthquake loci, collapsed buildings and the like.

In an embodiment of the present invention, the positioned composition includes extract of Hedychium, preferably Hedychium spicatum, preferably an extract of the root of Hedychium, preferably an essential oil of the root of Hedychium, especially the essential oil of the root of Hedychium spicatum. Such a composition is exceptionally suited for use in reducing the negative effects of malodor associated with cancer for example for such individuals as persons afflicted with cancer, persons undergoing medical treatment and health-care workers, especially such a composition is helpful in neutralizing ancipatory nausea (apparently triggered by the smell associated with locations such as hospitals, surgical wards, cancer wards, nursing homes and old age homes) of patients undergoing chemotherapeutic treatment.

In an embodiment of the present invention, the positioned composition includes extract of Menta, preferably an extract of Menta piperata, preferably an oil of Menta, preferably an essential oil of Menta, especially the essential oil of Menta piperata. Such a composition is exceptionally suited for use by pregnant women to reduce the negative effects of malodors that are known to irritate or otherwise cause undesired effects in pregnant women.

In an embodiment of the present invention, the positioned composition includes an extract of Citrus, preferably an extract of Citrus limonum, preferably an oil of Citrus, preferably an essential oil of Citrus, especially an essential oil of Citrus limonum. Such a composition is exceptionally suited for use in reducing the negative effects of malodor associated with fire and smoke for example caused by cigarette smoke, smoke, tobacco, burned oil or protein (flesh), charred protein (flesh) and the characteristic smells associated with fires.

In an embodiment of the present invention, the positioning is such that vapors emanating from the composition (or article) are inhaled by the individual concurrently with the inhaling of malodorous vapors emanated from the source of malodor.

In an embodiment of the present invention positioning the composition comprises positioning the composition in the vicinity of the source of the malodor or placing the composition at the location where the source of malodor is found. In a preferred embodiment, the composition is provided in an article including the composition. Preferred such articles include but are not limited to air-freshening products, decorative items (such as artificial flowers), sublimating air fresheners, fabrics, cloths, tissues, sponges, papers, and pads.

In an embodiment of the present invention, the positioning comprises positioning the composition in the vicinity of an olfactory organ of the individual. In such an embodiment, the composition is preferably provided as a smearable article and positioning the composition comprises smearing the smearable article in the vicinity of an olfactory organ of the individual, for example is on or near the filtrum of the individual.

In an embodiment of the present invention, positioning comprises contacting the source of malodor with the composition. Such an embodiment is exceptionally useful, for example when the source of malodor is an article of clothing, fabric, cloth or parts of a body such as the mouth, feet, hands and the like.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graph of odor detection as a function of cadaverine concentration comparing the ability to sense the odors of cadaverine and skatole when using a product of the present invention; and

FIG. 2 is a graph of the confidence rating of sensing a smell when using a product of the present invention.

### DESCRIPTION OF THE PREFERRED EMBODIMENTS

The present invention provides a method for ameliorating the negative effects of malodor on an individual. The present invention also provides compositions, a method of making the compositions and articles of manufacture that are useful in ameliorating the negative effects of malodors.

The amelioration achieved by the teachings of the present invention is achieved by affecting specific olfactory receptors in an individual thereby altering perception of the malodor. Although not wishing to be limited to any one theory, apparently a composition of the present invention achieves the perception-altering effect at the olfactory receptor sites by influencing the transporter protein thereby influencing the G-protein second messenger system, in such a way altering the perception of the malodor. The evidence indicates that components of the composition selectively bind to olfactory receptors in patterns that alter the perception of the odors normally associated with malodors. Thus in contrast with the prior art, the present invention does not ameliorate the effects of odors through masking but rather alters the perception of the odors by the brain by selectively binding to certain olfactory receptors.

In the broadest sense, the present invention provides a method of modifying the perception of odor by a subject by selectively binding olfactory receptors that in turn alter the perception of a malodor. It is generally known that the different olfactory receptors responsive to a malodor are influenced by a plurality of appropriate compounds, that is, any given malodor is detected by a plurality of different olfactory receptors. In prior art methods of odor masking it is likely that some of the many different olfactory receptors contributing to the sensing of a given malodor are blocked. The conception and implementation of the method of the present invention is innovative in selectively influencing a sufficient number of appropriate receptors so as to effectively modify the perception of odor to the point of ameliorating the discomfort arising from a specific malodor without completely blocking all sense of smell or even detection of the specific malodor.

The principles and uses of the present invention may be better understood with reference to the example and accompanying descriptions. Before explaining at least one embodiment of the invention in detail, it is to be understood that the invention is not limited in its application to the details set forth in the following description or exemplified by the example. The invention is capable of other embodiments or of being practiced or carried out in various ways. Also, it is to be understood that the phraseology and terminology employed herein is for the purpose of description and should not be regarded as limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Implementation of the methods of the present invention involves performing or completing selected tasks or steps manually, automatically, or a combination thereof.

As used herein the term “extract” includes but is not limited to products (e.g., tinctures, concretes, absolutes, oils, essential oils, oleoresins, terpenes, terpene-free fractions, distillates, glycolic extracts, lipo-soluble extracts, dry powder extracts, fluid extracts and residues) obtained from a source such as a plant or animal through an extraction process such as distillation, organic extraction, alcoholic extraction, aqueous extraction and solvent extraction.

The composition of the present invention is a novel mixture of known scents and/or fragrances and/or compounds and/or plant extracts characterized in that the combination is effective in altering the perception of at least one malodor by affecting at least one olfactory receptor, and as such are useful in implementing the method of the present invention. Embodiments of the composition are virtually scentless yet even such embodiments are effective in ameliorating the negative effect of malodors and stench, especially those associated with decomposing flesh, burned flesh, abscesses, urine, feces odors, ammonia-like odors, and amine and indole compounds produced during the putrefaction of flesh but also malodors characteristic of locations such as clinics, dental clinics, hospitals, surgical wards, mass graves, morgues, battlefields, abattoirs, industrial plants, sewers, earthquake loci, collapsed buildings, sewage processing plants, tanning plants, animal handling areas, barns, cancer wards, changing rooms, clarifiers, coal mines, composting sites, crematoria, crummy motels, diaper pails, dormitories, feed lots, garbage dumps, garbage processing plants, kennels, landfills, laundry rooms, leather processing plant, locker rooms, lumber mills meat processing plants, milking parlors, mines, mothers in law, nursing homes, old age homes, outhouses, paper mills, photographic products manufacturing plant, poultry processing plants, prisons, rendering plants, settling basins, sewage dewatering systems, sludge stations and sport centra and/or malodors characteristic of agriculture, amines, body odor, cigarette smoke, compost, dairy industry, gangrenous wounds, garbage, human feet, diapers, dirty laundry, halitosis, bad breath, lesions, livestock, manure, mercaptans, sewage, sludge, smoke, swine, tobacco, trash compactors, tumors, smoke, stale sweat, ulcers, unwashed humans, vomit, waste water, rotting proteins, decomposing proteins, burned proteins, viscera, amines produced during the putrefaction of proteins and indoles produced during the putrefaction of proteins and combinations thereof.

When a person inhales vapors released from the composition of the present invention concurrently with inhaling the vapors associated with malodor and stench, the person is aware of the smell of the malodor and stench but does not have any particularly strong reaction (e.g., retching, vomiting, fainting, nausea, fear, panic, disgust, nervousness, loss of concentration, shock, discomfort, desire to flee, headaches and associative memories). Thus, in contrast to prior art compositions and methods, the method and the composition of the present invention do not overcome odors through masking but alters the perception of the odors by the brain by selectively affecting certain olfactory receptors. Implementation of the present invention therefore allows an individual to perform a complex or sensitive task in the presence of malodor and stench with greater efficiency and with fewer ill-effects than without implementation.

An advantageous way of implementing the method of the present invention is by providing a composition where volatile components of the composition are the compounds that specifically affect the appropriate olfactory receptors. A preferred composition for implementing the method of the present invention comprises at least three plant extracts, at least four plant extracts or even at least five plant extracts, the plant extracts so chosen that volatile components of the composition are effective in affecting olfactory receptors so as to modify the perception of at least one malodor when inhaled. A preferred group from which to select the plant extracts consists of frankincense (preferably from Boswellia carteri), extract of roses (preferably flowers, preferably petals, preferably of Rosa damascena, especially those grown in Bulgaria, and preferably rose oil, rose oil absolute or a mixture thereof), extract of Cananga (preferably of Cananga odorata, preferably of the flower of Cananga, preferably the essential oil of the flower, especially ylang ylang) the essential oil of ylang ylang, specifically of Cananga odorata), extract of Piper (preferably of leaves, preferably of Piper auritum, preferably leaves of Piper auritum, preferably the essential oil), and extract of Bursera (preferably Bursera wood, preferably linaloe wood oil, especially Bursera glabrifolia and/or Bursera delpechiana).

Frankincense (olibanum) (CAS nr. 8016-36-2) is resin obtained from the leafy tree of Boswellia, such as Boswellia Thurifera and Boswelia Carteri. Frankincense is obtained by making a horizontal incision on the trunk of an appropriate tree and peeling off a strip of bark therefrom, from May until September. The incision is deepened over a period of months until the emanating sap hardens into globules Preferably, the total amount of frankincense in the composition of the present invention, when present, is greater than about 0.1% and up to 40% by weight of the composition.

Many different extracts of roses are known, especially of rose flowers, and especially of the petals of the flowers. Rose oil is the essential oil, generally obtained by distillation, of petals from the flower of Rosa damascena. Rose oil absolute (CAS nr. 8007-01-0) is a concentrated extract of petals from the flower of Rosa damascena, generally by a hexane extraction of the plant matter to form a concrete followed by ethanol or liquid CO₂ extraction of the concrete. Preferably, the total amount of rose extract, such as rose oil absolute or rose oil, in the composition of the present invention, when present, is greater than about 0.1% and up to 40% by weight of the composition.

A preferred extract of Cananga for implementing the teachings of the present invention is ylang ylang. Ylang ylang (CAS nr. 8006-81-3) is the essential oil extracted from the flower of Cananga odorata, generally steam or water distilled from freshly picked flowers. Preferably, the total amount of Cananga extract, such as ylang ylang, in a composition of the present invention, when present, is greater than about 0.1% and up to 40% by weight of the composition.

The genus Piper includes many leafy plants including Piper auritum known for having fragrant leaves. Mexican pepper leaf oil (Hoja Santa) is the essential oil extracted from leaves of Piper auritum. Preferably, the total amount of extract of a Piper plant, such as Mexican pepper leaf oil, in the composition of the present invention, when present, is greater than about 0.1% and up to 60% by weight of the composition.

Linaloe wood oil (CAS nr. 8006-86-8) is steam distilled from the wood of old and deliberately damaged trees of bursera, particularly bursera delpechiana and bursera glabrifloria. It is a common practice to include the fruits of the tree in the distillation to increase the yield of oil. Linaloe wood oil is a pale yellow or almost colorless liquid. Preferably, the total amount of extract of Bursera, such as linaloe wood oil, in the composition of the present invention, when present, is greater than about 0.1% and up to 22% by weight of the composition.

Other plant extracts useful in implementing the teachings of the present invention include but are not limited to: abelmoschus moschatus l. seed, abies alba mill absolute, abies alba mill oil from cones, abies balsamea l. mill. oil america, abies balsamea l. needle oil canada, abies balsamea l. needle oil terpeneless canada, abies picea lindl. needle oil, abies sibirica ledeb. needle oil siberia, abscess root (polemonium reptans linn.), absinthe oil (artemisia absinthium, absolue cire d'abeille, acacia abyssinica hochst. gum, acacia arabica gum, acacia concuine pods, acacia decurrens willd. var. dealbata absolute french, acacia decurrens willd. var. dealbata bark australia, acacia false bark (robinia pseudacacia), acacia farnesiana l. willd. absolute, acacia glaucophylla staud. gum, acacia gum (acacia senegal l. willd), acacia niopo seed amazon, achillea coronopifolia oil, achiflea millefolium l. oil, aconite england (aconitum napellus linn.), acorus calamus l. oil, adder's tongue america (erythronium americanum ker-gawl), adrue jamaica (cyperus articulatus linn.), aframomum melegueta rosc. k. schum. oil, aframomum melegueta rosc. oil, agal agal gum, agar gum, agar wood oil (aquilaria agallocha), agastache foeniculum oil, agathis australis leaf oil, agathis australis oil, agrimony hemp herb (eupatorium cannabinum linn.), ajowan seed oil (trachyspermum ammi), alder bark common (alnus glutinosa gaertn.), alder bark tag (alnus serrulata willd.), algae absolute brown, algae absolute brown, algae absolute green, algae absolute green, alkanet root (alkanna tinctoria tausch.), allium cepa l. oil, allium cepa l. oleoresin, allium sativum l. oil china, allium sativum l. oleoresin, allspice oil (pimenta officinalis lindl.), allspice oleoresin (pimenta officinalis lindl.), almond oil bitter (amygdalus communis linn. var. amara), almonds bitter, almonds sweet, alnus rubra bark (alnus serrulata willd.), aloe barbadensis leaves, aloe leaves, aloe vera linn. leaves, aloe wood gum, alpinia officinarum l. oleoresin, alpinia officinarum l. root oil, alstonia constricta f. muell. bark, alstonia scholaris, amber oil, ambrette seed (hibiscus abelmoschus), ambrette seed absolute (hibiscus abelmoschus), ambrette seed oil (hibiscus abelmoschus), ambrette seed resinoid (hibiscus abelmoschus), american greek valerian root, american mountain ash (pyrus americana d.c.), american pulsatilla, ammomum melegueta oil, ammoniacum gum (dorema ammoniacum d. don.), amygdalus communis linn. var. amara oil, amyris wood oil (amyris balsamifera), andropogon schoenanthus oil india, anethum graveolens l. oil america, anethum sows d.c. seed oil, angelica archangelica l. root, angelica archangelica l. root absolute, angelica archangelica l. root oil, angelica archangelica l. seed, angelica archangelica l. seed oil, angelica archangelica l. stem oil, angelica root (angelica archangelica), angelica root absolute (angelica archangelica), angelica root oil (angelica archangelica), angelica seed (angelica archangelica), angelica seed oil (angelica archangelica), angelica stem oil (angelica archangelica), angelica tree bark, angostura bark (galipea offincinalis hancock), anise hyssop oil (agastache foeniculum), anise oil, star anise oil terpeneless (illicium verum hook f.), anise oleoresin (pimpinella anisum), anise seed oil (pimpinella anisum), star anise seed oil (illicium verum hook f.) china, annatto seed (bixa orellana), anthemis nobilis flower oil, anthemis nobilis l. oil, apis mellifera l. absolute, apium graveolens l. leaf oil, apium graveolens l. oleoresin, apium graveolens l. seed oil india, apple balsam (poison) (momordica balsamina), apple bitter (poison) (citrullus colocynthis), apricot (prunus armeniaca), araroba powder (andira araroba aguiar.), araruta root (maranta arundinaceae), arbutus trailing (epigaea repens linn.), arbutus unede, areca nut, armeniaca vulgaris, armoise oil (artemisia vulgaris), armoracia lapathifolia gilib. oil, arnica flower oil (arnica montana), arnica flowers, arrowhead, arrowroot (maranta arundinaceae), artemisia absinthium l. oil, arternisia annua oil, artemisia dracunculus l. oil, artemisia pallens wall. oil, artemisia vulgaris oil, asafetida (ferula foetida regel.), asafetida absolute (ferula asafoetida), asafetida oil (ferula asafoetida), asarabacca (asarum europaeum), asarum canadense l. oil canada, asclepias syriaca willd., ash bark prickly (xanthoxylum americanum), ash prickly berries, asparagus (asparagus officinalis), aspic oil (lavandula latifolia v.l.), atis root (aconitum heterophyllum wall.), atlas cedarwood oil (cedrus atlanteca manetti) morocco, atractylis ovata l. root oil, attar of rose bulgaria, australian quinine (alstonia constricta f. muell.), bael fruit (aegle marmelos correa), balm leaves, balm lemon leaves (melissa officinalis), balm lemon oil (melissa officinalis), balmony (chelone glabra), balsam fir oil (abies balsamea l. mill.) america, balsam fir oleoresin (abies balsamea l. mill.), balsam fir oregon (pseudotsuga taxifolia), balsam peru (myroxylon pereirae klotzsch), balsam peru oil (myroxylon pereirae klotzsch), balsam peru resinoid (myroxylon pereirae klotzsch), balsamina (momordica balsamina), balsamodendron kafal absolute, balsamodendron kafal oil, balsamodendron kafal resinoid, balsamodendrum opobalsamum, balsamum meccae var. judiacum, balsamum tolutanum (myroxylon toluiferum), balsarnum americanum (myroxylon toluiferum), balsarnum gileadense, balsumodendron gileadensis, bamboo brier (aralia nudicaulis), baneberry (actaea spicata), barberry indian (berberis asiatica), barberry nepal (berberis aristata), barosma spp. oil, bartsia odontites, basil oil sweet (ocimum basilicum), basil sweet absolute (ocimum basilicum), basil sweet oleoresin (ocimum basilicum), bastard cabbage bark (andira inermis), bastard ipecacuanha (asclepias curas-savica), baume de la mecque (commiphora opobalsamum), bay leaves oil (pimenta racemosa mill.), bay leaves oil (pimenta racemosa) anise, bay leaves oil (pimenta racemosa) clove, bay leaves oil (pimenta racemosa) lemon, bay leaves oil terpeneless (pimenta racemosa mill.), bay leaves oleoresin (pimenta racemosa mill.), bay sweet oleoresin (laurus nobilis), bead tree (melia azadirachta), bearberry leaves (arctostaphylos uva-ursi), bearsfoot american (polymnia uvedalia), bedstraw hedge (galium molugo), beeswax absolute, beetroot, benzoin siam absolute (styrax tonkinensis craib ex hartwiss), benzoin siam resin (styrax tonkinensis craib ex hartwiss), benzoin siam resinoid (styrax tonkinensis craib ex hartwiss), benzoin sumatra absolute, benzoin sumatra resin, benzoin sumatra resinoid, berberis asiatica, bergamot mint oil (mentha citrata), bergamot oil (citrus aurantium l. subsp. bergamia wright et am.), bergamot oil bergaptene free (citrus aurantium l. subsp. bergamia wright et arm.), bergamot oil terpeneless (citrus aurantium l. subsp. bergamia wright et arn.), bergamot orange oil bergaptene free, bermuda arrowroot, betel leaf oil (piper betel), betel nut (areca catechu linn.), betula alba bud oil, betula alnus bark (alnus glutinosa gaertn.), betula pendula roth tar oil, bible leaf oil (chrysanthemum balsamita), bidens bipinnata linn., bigarade oil (citrus bigaradia), bikh root (aconitum laciniatum staph.), birch black oil (betula lenta) america, birch bud oil (betula alba), birch oil sweet (betula lenta) america, birch tar oil (betula pendula roth), bish root, bitter almond oil (amygdalus communis linn. var. amara), bitter almonds (amygdalus communis linn. var. amara), bitter ash (picraena excelsa swartz), bitter cucumber (citrullus colocynthis), bitter orange leaf oil (citrus aurantium), bitter orange peel oil (citrus aurantium) brazil, bitter orange peel oil (citrus aurantium) china, bitter orangeflower oil (citrus bigaradia), bixa orellana l. seed, black caraway seed oil (nigella sativa), black currant bud absolute (ribes nigrum), black pepper absolute (piper nigrum), black pepper oil (piper nigrum) india, black pepper oleoresin (piper nigrum), black spruce oil (picea mariana mill.), black wattle bark (acacia decurrens willd. var. dealbata), blood weed (asclepias curas-savica), blue bells root (polemonium reptans linn.), blue chamomile flower oil (matricaria chamomilla), blue rocket (aconitum napellus linn.), blunt leaved everlasting (gnaphalium polycephalum), bois de rose oil (aniba rosaeodora var. amazonia ducke) brazil, bois de rose oil terpeneless (aniba rosaeodora ducke), boldo leaf oil (peumus boldus mol.), boldus leaf oil (peumus boldus mol.), boronia absolute (boronia megastigma nees), boronia megastigma nees absolute, boswellia carteri birdw. oil, brachyleana hutchinsii wood oil, branched asphodel, broom absolute (spartium junceum), buchu leaf oil (barosma betulina bartl & wendl.), bugbane (actaea spicata), bulnesia sarmienti oil, bur marigold (bidens tripartita linn.), bursera delpechiana poiss and other burs era spp. wood oil mexico, bursera spp. wood oil mexico, butterfly milkweed (asclepias tuberosa), butterfly weed, cabbage tree of south america (andira inermis), cabreuva wood oil (myrocarpus frondosus & m. fastigiatus), cade oil (juniperus oxycedrus), cajuput oil (melaleuca leucadendron), calamus oil (acorus calamus), camphor oil white japanese (cinnamomum camphora l. nees se eberm.), canadian hemlock oil, cananga odorata hook absolute, cananga odorata hook. f. and thomas. oil, cananga odorata hook. f. and thomas. oil java, cananga oil (cananga odorata hook. f. and thomas.) java, cananga oil terpeneless, canarium indicum l. absolute, canarium indicum l. oil, canarium indicum l. resinoid, canarium spp. gum, canella alba murr. bark oil, canella bark oil (canella alba), capsicum oleoresin (capsicum annuum l. var. longum sendt), caraway seed oil (carum carvi), caraway seed oleoresin (carum carvi), cardamom absolute (elletaria cardamomum l. maton), cardamom liquid resin (elletaria cardamomum l. maton), cardamom oleoresin (elletaria cardamomum l. maton), cardamom seed oil (elletaria cardamomum l. maton) west india, cardoon artichoke (scolymus cardunculus), carnation absolute (dianthus caryophyllus), carrot seed oil (daucus carota sativa), carrot weed oil (daucus carota sativa), carrotweed oil, carum ajowan oil, carum carvi l. oil, carum carvi l. seed oleoresin, carum copticum benth. et hook oil, cascarilla bark oil (croton spp.), cassia bark oil (cinnamomum cassia blume) china, cassia leaf oil (cinnamomum cassia blume) china, cassia oleoresin (cinnamomum cassia blume), cassie absolute (acacia farnesiana l. willd.), cassis bud absolute (ribes nigrum), cassis bud oil (ribes nigrum), catsfoot (gnaphalium polycephalum), cayenne oleoresin, cedarleaf oil (thuja occidentalis) canada, cedarleaf oil terpeneless (90-95% thujone) (thuja occidentalis), cedarleaf oil western red (thuja plicata donn ex. d. don), cedarwood absolute atlas (cedrus atlanteca manetti), cedarwood oil (cedrus deodara (roxb.) loud.) himalaya, cedarwood oil atlas (cedrus atlanteca manetti) morocco, cedarwood oil chinese, cedarwood oil port orford (chamaecyparis lawsoniana), cedarwood oil red america, cedarwood oil texas (juniperus mexicana) america, cedarwood oil Virginian (juniperus virginiana l.) america, cedrat peel oil (citrus medica var. bajoura), cedrat petitgrain oil (citrus medica var. bajoura), cedro oil terpeneless, cedrus atlanteca manetti absolute, cedrus atlanteca manetti oil morocco, cedrus deodara (roxb.) loud. oil himalaya, celery leaf oil (apium graveolens), celery seed oil (apium graveolens l.) india, celery seed oleoresin (apium graveolens), chamaecyparis lawsoniana wood oil, chamaecyparis obtusa l. leaf oil, chamaecyparis obtusa l. root oil, chamomile flower oil (anthemis nobilis) england, chamomile flower oil german (matricaria chamomilla), chamomile flower oil hungarian (matricaria chamomilla), chamomile flower oil roman (anthemis nobilis), chamomile oil (ormenis multicaulis) morocco, champaca absolute (michelia champaca), champaca flower oil, cheese renning (galium verum), chenopodium ambrosioldes var. anthelminthicum oil america, cherry laurel oil (prunus laurocerasus), chinese artichoke (stachys sieboldii), chipotle chili oleoresin, chrysanthemum balsamita l. oil, chrysatobine (andira araroba aguiar.), cinnamomum camphora l. nees se eberm. leaf oil, cinnamomum camphora l. nees se eberm. wood oil, cinnamomum cassia blume bark oil china, cinnamomum cassia blume leaf oil china, cinnamomum cassia blume oleoresin, cinnamomum cecidodaphne meissn berry oil, cinnamomum glaucescens nees berry oil, cinnamomum zeylanicum blume bark oil ceylon, cinnamomum zeylanicum blume leaf oil ceylon, cinnamomum zeylanicum blume twig oil india, cinnamon bark oil (cinnamomum zeylanicum blume) ceylon, cinnamon leaf oil ceylon, cinnamon oleoresin, cinnamon twig oil (cinnamomum zeylanicum blume) india, cire d'abeille absolute, cistus absolute (cistus ladaniferus), cistus creticus l. resin, cistus ladaniferus l. absolute, cistus ladaniferus l. oil, cistus oil (cistus ladaniferus), cistus spp. absolute resin, cistus spp. gum, cistus spp. oil, cistus spp. resinoid, citronella oil ceylon (cymbopogon nardus rendle), citronella oil chinese, citronella oil java (cymbopogon winterianus jowitt), citrus aurantifolia (christmuan) swingle oil terpeneless, citrus aurantifolis (christman) swingle expressed oil florida, citrus aurantifolis swingle distilled oil, citrus aurantium l. absolute france, citrus aurantium l. oil, citrus aurantium l. peel oil, citrus aurantium l. peel oil bitter brazil, citrus aurantium l. peel oil bitter china, citrus aurantium l. peel oil bitter terpeneless, citrus aurantium l. subsp. amara absolute morocco, citrus aurantium l. subsp. bergamia wright et am. oil, citrus aurantium l. subsp. bergamia wright et am. oil bergaptene free, citrus aurantium l. var amara water absolute, citrus aurantium oil paraguay, citrus aurantium oil terpeneless paraguay, citrus limon l. burm. f. leaf oil, citrus limon l. burm. oil california, citrus limon l. burm. f. oil distilled, citrus limon l. burm. f oil terpeneless, citrus maxima oil, citrus medica var. bajoura oil, citrus medica var. bajoura peel oil, citrus paradisi macf. oil california, citrus paradisi oil terpeneless, citrus reticulata blanco var. mandarin leaf oil, citrus reticulata blanco var. mandarin oil italy, citrus reticulata blanco var. mandarin oil terpeneless, citrus reticulata oil dancy, citrus reticulata oil terpeneless, citrus sinensis l. osbeck peel oil cold pressed brazil, citrus sinensis l. osbeck peel oil cold pressed california, citrus sinensis l. osbeck peel oil cold pressed china, citrus sinensis l. osbeck peel oil cold pressed florida, citrus sinensis l. osbeck peel oil cold pressed valencia, citrus sinensis l. osbeck peel oil distilled, city avens (geum urbanum linn.), clary sage absolute (salvia sclarea), clary sage oil (salvia sclarea) france, clary sage resin (salvia sclarea), clary sage resinoid, clove bud absolute (eugenia caryophyllata), clove bud oil (eugenia caryophyllata), clove bud oleoresin (eugenia caryophyllata), clove leaf oil (eugenia caryophyllata), clove leaf oil terpeneless (eugenia caryophyllata), clove root (geum urbanum inn.), clove stem oil (eugenia caryophyllata) india, cocoa oleoresin, coconut absolute (cocos nucifera), cocos nucifera l. absolute, cognac oil green, cognac oil white, colewort (geum urbanum linn.), commiphora erythraea var. glabrescens engle absolute, commiphora erythraea var. glabrescens engle oil, commiphora erythraea var. glabrescens engle resinoid, commiphora erythraea var. glabrescens engler resin, commiphora spp. absolute, commiphora spp. gum, commiphora spp. oil, commiphora spp. resin, commiphora spp. resinoid, common alder bark (alnus glutinosa gaertn.), common ash (fraxinus excelsior), convallaria majalis l. absolute, copaiba, copaiba balsam (copaifera langsdorffi), copaiba balsam oil (south american spp. of copaifera), copaifera langsdorffi, copaifera langsdorffi oil, coriander leaf absolute (coriandrum sativum), coriander leaf oil (coriandrum sativum), coriander oleoresin (coriandrum sativum), coriander seed absolute (coriandrum sativum), coriander seed oil (coriandrum sativum), coriandrum sativum l. oil, coriandrum sativum l. oleoresin, coriandrum sativum leaf oil, cornmint oil (mentha arvensis) paraguay, cornmint oil terpeneless (mentha arvensis), costmary oil (chrysanthemum balsamita), costus root absolute (saussurea lappa clarke), costus root oil (saussurea lappa clarke), costus root resinoid (saussurea lappa clarke), crocus sativus l. resinoid, crocus sativus oil, croton spp. bark oil, crowfoot (anemone nemorosa linn.), cryptocaryo massoio oil, cryptomeria japonica d. don. wood oil, cubeb oil (piper cubeba), cubeb oleoresin (piper cubeba), cumin oleoresin (cuminum cyminum), cumin seed oil (cuminum cyminum), cuminum cyminum l. oil, cuminum cyminum l. oleoresin, cupressus sempervirens l. absolute, cupressus sempervirens l. oil, curacao peel oil (citrus aurantium), curcuma longa l. oleoresin, curcuma longa l. root oil, curcuma longa l. root oleoresin, cure all (geum rivale linn.), cymbopogon citratus dc and cymbopogon flexuosus oil east indian, cymbopogon martini roxb. oil india, cymbopogon martini roxb. stapf oil india, cymbopogon martini var. sofia oil, cymbopogon nardus rendle oil, cymbopogon stapf. oil egypt, cymbopogon winterianus jowitt oil, cyperiol (cyperus rotundus l. oil), cyperus oil (cyperus scariosus l.), cyperus root oil (cyperus rotundus), cyperus root oil (cyperus scariosus), cyperus rotundus l. root oil, cyperus scariosus l. root oil, cypress absolute (cupressus sempervirens), cypress oil (cupressus sempervirens), dacrydium fanklinii wood oil, damiana false, darlahad (berberis aristata), daucus carota l. seed oil, daucus carota l. weed oil, davana oil (artemisia pallens wall.), deertongue absolute (liatris odoratissima (walt.) willd.), deertongue leaves (liatris odoratissima (walt) willd.), deertongue oleoresin (liatris odoratissima (walt.) willd.), deodar cedar oil himalaya, devil tree bark, devil's dung, dianthus caryophyllus absolute, dill herb oil (anethum graveolens) america, dill seed oil (anethum graveolens), dill weed oil (anethum graveolens) america, dipterocarpus spp. balsam, dipterocarpus spp. oil, dipteryx odorata bean absolute, dipteryx odorata oleoresin, dita bark, dog's tooth violet (etlironium americanum ker-gawl), dorema ammoniacum & don. gum, dryas octopetala linn, dyer's bugloss root (alkanna tinctoria tausch.), easter flower (anemone pulsatiila linn.), eastern spruce oil, edeltanne needle oil (abies alba mill), elecampane absolute (inula helenium), elecampane oil (inula helenium), elecampane root oil (inula helenium), elemi absolute (canarium indicum), elemi gum (canarium indicum), elemi oil (canarium indicum), elemi resinoid (canarium indicum), elletaria cardamomum l. maton absolute, elletaria cardamomum l. maton seed oil west india, encens absolute, erigeron canadensis l. oil, erigeron oil (erigeron canadensis), estragon oil (artemisia dracunculus), eucalyptus citriodora oil, eucalyptus dives type oil, eucalyptus dives var. “a” oil, eucalyptus dives var. “c” oil, eucalyptus globulus labille oil, eucalyptus oil (eucalyptus globulus labille), eugenia caryophyllata l. bud absolute, eugenia caryophyllata l. bud oil, eugenia caryophyllata l. bud oleoresin, eugenia caryophyllata l. leaf oil, eugenia caryophyllata l. leaf oil terpeneless, eugenia caryophyllata l. stem oil india, eupatorium cannabinum linn., european silver fir needle oil (abies alba mill), everlasting absolute (helichrysum angustifolium), everlasting oil (helichrysum angustifolium), evernia furfuraceae spp. absolute, evernia firfinaceae spp. resinoid, evernia prunasti spp. oil, evernia prunasti spp. resin, false damiana, false jacob's ladder root (polemonium reptans linn.), featherfew leaf oil, fennel absolute sweet (foeniculum vulgare mill. var. dulce d.c.), fennel oil bitter (foeniculum vulgare miller), fennel oil sweet (foeniculum vulgare mill. var. dulce d.c.), fennel oleoresin sweet (foeniculum vulgare mill. var. dulce d.c.), fennel roman oil (foeniculum vulgare mill. var. dulce d.c.), fennel seed oil bitter (foeniculum vulgare miller), fennel seed oil sweet (foeniculum vulgare mill. var. dulce d.c.), fenugreek absolute (trigonella foenum graecum), fenugreek oleoresin (trigonella foenum graecum), fenugreek resinoid (trigonella foenum graecum), ferula asafoetida l. absolute, ferula asafoetida l. oil, ferula galbaniflua absolute, ferula galbaniflua oil, ferula galbaniflua oil terpeneless, ferula galbaniflua oleoresin, ferula galbaniflua resinoid, fever bark (alstonia constricta f. muell.), fever bush (prinos verticillatus linn.), fir balsam absolute (abies alba mill), fir balsam oleoresin oregon, fir balsam oregon (pseudotsuga taxifolia), fir needle oil (abies balsamea) canada, fir needle oil terpeneless (abies balsamea) canada, fir siberian oil (abies sibirica ledeb.) siberia, fir silver oil (abies balsamea l. mill.) america, flake manna (fraxinus omus), fleabane oil (erigeron canadensis), foeniculum vulgare mill. var. dulce d.c. absolute, foeniculum vulgare mill. var. dulce d.c. oil, foeniculum vulgare mill. var. dulce d.c. oleoresin, foeniculum vulgare miller oil, food of the gods, fragrant cubweed, fragrant everlasting (gnaphalium polycephalum), frangipanni pink absolute (plumiera rubra), frankincense gum (boswellia carterii birdw.) somalia, frankincense oil (boswellia carterii birdw.), franincense resin (boswellia carterii birdw.) samolia, fraxinus excelsior l., fraxinus omus l., friar's cap (aconitum napellus linn.), fucus vesiculosus et serratus absolute, galangal root oil (alpinia officinarum), galangal root oleoresin (alpinia officinarum), galbanum absolute (ferula galbaniflua), galbanum oil (ferula galbaniflua), galbanum oil terpeneless (ferula galbaniflua), galbanum oleoresin (ferula galbaniflua), galbanum resinoid (ferula galbaniflua), galipea offincinalis hancock bark, galium molugo l., garden angelica root (angelica archangelica), garden angelica seed (angelica archangelica), garden arrach (atriplex hortensis), garden beet, garden orache (atriplex hortensis), garden rosemary oil (rosmarinus officinalis) spain, gardenia absolute (gardenia grandiflora), gardenia grandiflora l. absolute, garlic oil (allium sativum) china, garlic oleoresin (allium sativum), gaultheria procumbens l. oil china, genet absolute (spartium junceum), genievre baies oil (juniperus communis), geranium absolute (pelargonium graveolens), geranium oil (cymbopogon stapf.) egypt, geranium oil (pelargonium graveloens) africa, geranium oil (pelargonium graveolens) bourbon, geranium oil chinese, geranium oil moroccan, geranium oil terpeneless (pelargonium graveolens), geranium rose oil (pelargonium graveolens), geranium turkish oil (cymbopogon martini roxb. stapf) india, ginger absolute (zingiber officinale rosc.), ginger oleoresin (zingiber officinale rosc.), ginger oleoresin (zingiber officinale rosc.) africa, ginger root oil (zingiber officinale rosc.) cochin, ginger root oil terpeneless (zingiber officinale rosc.) cochin, gingergrass oil (cymbopogon martini var. sofia), goa powder (andira araroba aguiar.), goat's arrach, goldy star (geum urbanum linn.), gomenol oil (melaleuca quinquenercia (cav.) s t blake), grains of paradise oil (aframomum melegueta rosc. k. schum.), grapefruit oil c.p. (citrus paradisi macf.) california, grapefruit oil terpeneless (citrus paradisi macf.), gravel plant (epigaea repens linn.), greek valerian american root (polemonium reptans linn.), ground laurel (epigaea repens linn.), guaiacwood oil (bulnesia sarmienti), guarana gum (paullinia cupana hbk), gum acacia (acacia senegal l. willd), gum ammoniac (dorema ammoniacum d. don.), gum arabic (acacia senegal l. willd), gum senegal (acacia senegal l. willd), gurjun balsam (dipterocarpus spp.), gurjun balsam oil (dipterocarpus spp.), halberd-leaved arrach (atriplex hastata), halberd-leaved wild orache (atriplex hastata), hay absolute (lolium perenne), hazelnut oleoresin, hazelwort (asarum europaeum), hedeoma oil (mentha pulegium), hedera helix l. absolute, hedge basil, hedge calamint, helichrysum angustifolium dc. absolute, helichrysum angustifolium dc. oil, helichrysum angustifolium leaf oil, heliotrope absolute (heliotropium arborescens), heliotropium arborescens l. absolute, hemlock oil (tsuga canadensis), hemp agrimony herb (eupatorium cannabinum linn.), herb bennet (geum urbanum linn.), herb christopher (actaea spicata), hercules club bark (angelica tree), hercules club berries (angelica tree), hiba wood oil (thujopsis dolabrata), hibiscus abelmoschus l. seed, hibiscus abelmoschus l. seed absolute, hibiscus abelmoschus l. seed oil, hindi nimba oil india, hinoki leaf oil (chamaecyparis obtusa), hinoki root oil (chamaecyparis obtusa), ho leaf oil (cinnamomum camphora l. nees se eberm.), ho. wood oil (cinnamomum camphora l. nees se eberm.), holy rope (eupatorium cannabinum limn.), holy tree (melia azadirachta), hop oil (humulus lupulus), hop tree, horseradish oil (armoracia lapathifolia gilib.), humulus lupulus l. oil, huon pine wood oil (dacrydium frankinii), hyacinth absolute (hyacinthus orientalis), hyacinthus orientalis l. absolute, hyssop anise oil (agastache foeniculum), hyssop oil (hyssopus officinalis), hyssopus officinalis l. oil, ilex paraguariensis st. hil. absolute, illicium verum hook f. seed oil china, immortelle absolute (helichrysum angustifolium), immortelle leaf oil (helichrysum angustifolium), immortelle oil (helichrysum angustifolium), indian aconite root (aconitum laciniatum staph.), indian arrowroot (maranta arundinaceae), indian bael (aegle marmelos correa), indian chocolate (geum rivale linn.), indian gum, indian lilac tree (melia azadirachta), indian posy (gnaphalium polycephalum), indian valerian root oil false (nardostachys jatamansi), inula helenium l. absolute, inula helenium l. oil, is'-ze-kn, ivy leaf absolute (hedera helix), jacob's ladder false root (polemonium reptans limn.), jalapeno oleoresin, jamaica quassia (picraena excelsa swartz), jambu oleoresin (spilanthes acmelia oleracea), japanese cryptomeria wood oil (cryptomeria japonica d. don.), jasmin absolute chassis (jasminum grandiflorum), jasmin absolute concrete (jasminum grandiflorum) italy, jasmin absolute pommade (jasminum grandiflorum), jasmin oil (jasminum grandiflorum), jasminum grandiflorum l. absolute chassis, jasminum grandiflorum l. absolute italy, jasminum grandiflorum l. absolute pommade, jasminum grandiflorum l. oil, jerusalem artichoke (helianthus tuberosus), jonquil absolute (narcissus jonquilla), juniper absolute (juniperus communis), juniper needle oil (juniperus communis), juniper tar oil (juniperus oxycedrus), juniperberry absolute (juniperus communis), juniperberry oil (juniperus communis), juniperberry oil terpeneless (juniperus communis), juniperberry oleoresin, juniperus communis l. absolute, juniperus communis l. oil, juniperus communis l. oil terpeneless, juniperus mexicana oil america, juniperus oxycedrus l. tar oil, juniperus phoenicea l. oil, juniperus sabina oil, juniperus virginiana l. oil america, kauri-copal leaf oil (agathis australis), kauri-copal oil (agathis australis), keruing balsam (dipterocarpus spp.), keruing oil (dipterocarpus spp.), kewda absolute (pandanus odoratissimus), kidney bean (phaceolus vulgaris), king's spear, labdanum absolute resin (cistus spp.), labdanum gum (cistus spp.), labdanum oil (cistus spp.), labdanum resin (cistus creticus), labdanum resinoid (cistus spp.), lady's bedstraw (galium verum), lathyrus latifolius absolute, laurel berry absolute (laurus nobilis), laurel berry oil (laurus nobilis), laurel leaf absolute (laurus nobilis), laurel leaf oil (laurus nobilis), laurus nobilis l. berry absolute, laurus nobilis l. berry oil, laurus nobilis l. leaf absolute, laurus nobilis l. leaf oil, laurus nobilis l. oleoresin, lavandin absolute (lavandula hydrida), lavandin oil (lavandula hydrida) abrialis, lavandin water absolute (lavandula hydrida), lavandula angustifolia absolute bulgaria, lavandula angustifolia absolute france, lavandula angustifolia oil, lavandula angustifolia oil bulgaria, lavandula angustifolia oil terpeneless, lavandula hydrida absolute, lavandula hydrida oil abrialis, lavandula hydrida water absolute, lavandula latifolia v.l. absolute, lavandula latifolia v.l. oil, lavandula officinalis oil france, lavender absolute (lavandula angustifolia) bulgaria, lavender absolute (lavandula angustifolia) france, lavender oil (lavandula angustifolia), lavender oil (lavandula angustifolia) bulgaria, lavender oil (lavandula officinalis) france, lavender oil 40/42%, lavender oil terpeneless (lavandula angustifolia), lavender spike absolute (lavandula latifolia v.l.), lavender spike oil (lavandula latifolia v.l.), lawson false cypress wood oil, leaf cup (polymnia uvedalia), leek oil (allium porum), lemon balm leaves (melissa officinalis), lemon balm oil, lemon leaf oil (citrus limon l. burin. f.), lemon oil distilled (citrus limon l. burm. f.) spain, lemon oil expressed (citrus limon l. burm. f.) california, lemon oil terpeneless (citrus limon l. burm. f.), lemongrass oil (cymbopogon citratus dc and cymbopogon flexuosus) east indian, lentisk oil (pistacia lentiscus), lentisque gum (pistacia lentiscus), leopard's bane flowers (arnica montana), leopard's bane root (arnica montana), levisticum officinale koch absolute, levisticum officinale koch herb oil, levisticum officinale koch leaf oil, levisticum officinale koch oleoresin, levisticum officinale koch root oil, liatris odoratissima (walt.) willd. absolute, liatris odoratissima (walt.) willd. leaves, liatris odoratissima (walt.) willd. oleoresin, lilac absolute (syringa vulgaris), lilium candidum l. absolute, lily absolute (lillum candidum), lily of the valley absolute (convallaria majalis), lime oil distilled (citrus aurantifolis swingle) mexico, lime oil distilled terpeneless, lime oil expressed (citrus aurantifolis (christman) swingle) florida, lime oil expressed terpeneless (citrus aurantifolia (christman) swingle), linaloe wood oil (bursera delpechiana poiss and other burs era spp.) mexico, lippia citriodora absolute france, lippia citriodora cymbopogon spp. oil france, litsea cubeba oil china, litsea cubeba oil terpeneless, locust tree bark (robinia pseudacacia), lolium perenne l. absolute, lovage herb oil (levisticum officinale koch), lovage leaf oil (levisticum officinale koch), lovage oleoresin (levisticum officinale koch), lovage root absolute (levisticum officinale koch), lovage root oil (levisticum officinale koch), love-lies-bleeding (amaranthus hypochondriacus linn.), mace absolute (myristicia fragrans houtt.), mace oil (myristicia fragrans houtl), mace oleoresin (myristicia fragrans houtt.), maid's hair (galium verum), majorana hortensis moench (origanum ma jorana l.) oil, mandarin oil (citrus reticulata blanco var. mandarin) italy, mandarin petitgrain oil (citrus reticulata blanco var. mandarin), mandarin petitgrain oil terpeneless (citrus reticulata blanco var. mandarin), manna ash (fraxinus omus), margosa (melia azadirachta), margosa oil india, marigold absolute (tagetes minuta l.) egypt, marigold absolute (tagetes patula l.) india, marigold nodding (bidens cernua), marigold oil (tagetes glandulifera l.) mexico, marigold oil (tagetes minuta l.) egypt, marigold oil (tagetes patula l.) india, marjoram oil (thymus mastichina) spain, marjoram oil sweet (majorana hortensis moench (origanum majorana l.)), marjoram oleoresin (marjorana hortensis moench (origanum marorana l.)), marjorana hortensis moench (origanum marjorana l.) oleoresin, massoia bark oil (cryptocaryo massoio), mastic absolute (pistacia lentiscus), mastic gum resin (pistacia lentiscus), mastic oil (pistacia lentiscus), mastic resinoid (pistacia lentiscus), mate absolute (ilex paraguariensis st. hil.), matricaria chamomilla l. oil, may flower (epigaea repens linn.), meadow anemone (anemone pulsatilla linn.), melaleuca altemifolia oil australia, melaleuca cajuputi powell oil, melaleuca leucadendron l. oil, melaleuca quinquenercia (cav.) s t blake oil, melaleuca viridiflora oil, melasol (melaleuca alternifolia) australia, melilot oleoresin, melilotus oleoresin, melissa oil (melissa officinalis), mentha arvensis oil paraguay, mentha arvensis oil terpeneless, mentha cardiaca oil, mentha citrata oil, mentha piperita l. oil america, mentha piperita oil terpeneless america, mentha pulegium l. oil, mentha spicata absolute, mentha spicata oil terpeneless, michelia alba flower oil, michelia champaca l. absolute, mignonette absolute pomade, milfoil oil (achillea millefolium), mimosa absolute (acacia decurrens willd. var. dealbata) french, mistletoe absolute (viscum album), monkshood (aconitum napellus linn.), mountain ash (pyrus aucuparia gaertn.), mountain avens (dryas octopetala linn.), mountain pepper oil, mountain pink (epigaea repens linn.), mountain spinach (atriplex hortensis), mountain tobacco flowers (arnica montana), mountain tobacco root (arnica montana), mousse d'arbre absolute (evernia furfuraceae spp.), mousse de chene absolute (evernia prunasti spp.), mugo pine oil, mugwort oil (artemisia vulgaris), muhuhu wood oil (brachyleana hutchinsii), musk seed (abelmoschus moschatus), mutterkraut leaf oil, myrcia leaves oil, myristica fragrans flower oil, myristica fagraus houtt. absolute, myristica fragrans houtt. oil india, myristica fragrans houtt. oil terpeneless, myristica fragrans houtt. oleoresin, myristica fragrans leaf oil, myristicia fragrans houtt absolute, myristicia fragrans houtt. oil, myristicia fragrans houtt. oleoresin, myrocarpus frondosus & m. fastigiatus wood oil, myroxylon balsanum l. absolute, myroxylon pereirae klotzsch oil, myroxylon toluiferum l., myrrh absolute (commiphora spp.), myrrh gum (commiphora spp.), myrrh oil (commiphora spp.), myrrh resin (commiphora spp.), myrrh resinoid (commiphora spp.), myrtle oil (myrtus communis), myrtus communis l. oil, narcissus absolute (narcissus spp.), narcissus jonquilla l. absolute, narcissus spp. absolute, nard root oil, nardostachys jatamansi root oil, neem, neem oil (azadarachta indica) india, neemba oil india, nepal aconite root (aconitum laciniatum staph.), neroli bigarade oil (citrus bigaradia), netchweed, niaouli oil (melaleuca quinquenercia (cav.) s t blake), nicotiana spp. flower absolute, nicotiana spp. leaf absolute, nigella sativa l. seed oil, nodding avens (geum rivale linn.), nodding begger-ticks, none so pretty (gnaphalium polycephalum), norway pine oil (pinus sylvestris), norway spruce oil (picea abies), nutmeg absolute (myristica fragrans houtt.), nutmeg flower oil (myristica fragrans), nutmeg leaf oil (myristica fragrans), nutmeg oil (myristica fragrans houtt.) india, nutmeg oil terpeneless (myristica fragrans houtt.), nutmeg oleoresin (myristica fragrans houtt.), oakmoss absolute (evernia prunasti spp.), oakmoss oil (evernia prunasti spp.), oakmoss resin (evernia prunasti spp.), ocimum basilicum l. absolute, ocimum basilicum l. oil, ocimum basilicum l. oleoresin, ocimum minumum l., ocotea cymbarum oil, old field balsam (gnaphalium polycephalum), olea fragrans absolute, olibanum absolute (boswellia carterii birdw.), olibanum gum (boswellia carterii birdw.) somalia, olibanum oil (boswellia carterii birdw.), olibanum resin (boswellia carterii birdw.) samolia, olibanum resin samolia, olibanum resinoid (boswellia carterii birdw.), onion oil (allium cepa), onion oleoresin (allium cepa), ophthalmic barberry (berberis aristata), opoponax absolute (balsamodendron kafal), opoponax absolute (commiphora erytraea var. glabrescens engle), opoponax oil (balsamodendron kafal), opoponax oil (commiphora erythraea var. glabrescens engle), opoponax resin (commiphora erythraea var. glabrescens engler), opoponax resinoid (balsamodendron kafal), opoponax resinoid (commiphora erythraea var. glabrescens engle), orange leaf oil bitter (citrus aurantium), orange peel oil bitter (citrus aurantium) brazil, orange peel oil bitter (citrus aurantium) china, orange peel oil bitter terpeneless (citrus aurantium), orange peel oil sweet c.p. (citrus sinensis l. osbeck) brazil, orange peel oil sweet c.p. (citrus sinensis l. osbeck) california, orange peel oil sweet c.p. (citrus sinensis l. osbeck) china, orange peel oil sweet c.p. (citrus sinensis l. osbeck) florida, orange peel oil sweet c.p. (citrus sinensis l. osbeck) valencia, orange peel oil sweet distilled (citrus sinensis l. osbeck), orange peel oil sweet terpeneless (citrus sinensis l. osbeck), orangeflower absolute (citrus aurantium l. var amara) morocco, orangeflower bitter oil (citrus aurantium l. var amara), orangeflower water absolute (citrus aurantium l. var amara), orchanet root (alkanna tinctoria tausch.), oregano oleoresin (origanum vulgare), origanum marjorana l. oleoresin (marjorana hortensis moench (origanum marjorana l.)), origanum oil (thymus capitatus l. hoffmanns & link) spain, ormenis multicaulis oil morocco, orris absolute (iris pallida lam.), orris root absolute (iris pallida lam.), orris root resinoid (iris pallida lam.), osmanthus absolute (olea fragrans), palmarosa oil (cymbopogon martini roxb. stapf) india, pandanus odoratissimus absolute, paprika oleoresin, parsely oleoresin, parsley leaf oil (petroselinum crispum mill.), parsley seed oil (petroselinum savitum umbelliferae), parsley seed oleoresin, partridge vine oil (gaultheria procumbens) china, passe flower (anemone pulsatilla linn.), passiflora incarnata l. absolute, passionflower absolute (passiflora incarnata), patchouli absolute (pogostemon patchouli), patchouli oil (pogostemon patchouli), paullinia cupana hbk gum, pearl barley (hordeum distichon), pelargonium graveolens absolute, pelargonium graveolens l'her. oil, pelargonium graveolens oil africa, pelargonium graveolens oil bourbon, pelargonium graveolens oil terpeneless, pelargonium spp. oil, pennyroyal oil (mentha pulegium), pepper oil black (piper nigrum) india, pepper oil white (piper nigrum), pepper oleoresin black (piper nigrum), pepper oleoresin white (piper nigrum), pepper red oleoresin, pepper tree berry oil (schinus molle), pepper tree leaf oil (schinus molle l.), peppermint oil (mentha piperita) america, peppermint oil terpeneless (mentha piperita) america, perilla frutescens oil, perilla oil (perilla frutescens), perlatum (hordeum distichon), pem balsam (myroxylon pereirae klotzsch), peru balsam oil (myroxylon pereirae klotzsch), peru balsam resinoid (myroxylon pereirae klotzsch), petitgrain absolute (citrus aurantium) france, petitgrain bigarade oil (citrus aurantium), petitgrain bigarade surfleurs d'oranger oil, petitgrain lemon oil (citrus limon l. burm. f), petitgrain lemon oil terpeneless, petitgrain mandarin oil (citrus reticulata blanco var. mandarin), petitgrain mandarin oil terpeneless (citrus reticulata blanco var. mandarin), petitgrain oil (citrus aurantium) paraguay, petitgrain oil terpeneless (citrus aurantium) paraguay, petroselinum crispum mill. leaf oil, petroselinum savitum umbelliferae seed oil, petty mugget (galium verum), peumus boldus mol. leaf oil, phaceolus vulgaris, picea abies oil, picea mariana mill. oil, picea rubens sarg. oil, picea sitchensis oil, picraena excelsa swartz, pimenta acris kostel leaves oil, pimenta acris kostel leaves oil terpeneless, pimenta acris kostel leaves oleoresin, pimenta leaf oil (pimenta officinalis lindl.), pimenta officinalis lindl. oil, pimenta officinalis lindl. oleoresin, pimenta officinalis myrtaceae berry absolute, pimenta oil (pimenta officinalis lindl.), pimenta racemosa leaves oil anise, pimenta racemosa leaves oil clove, pimenta racemosa leaves oil lemon, pimento berry absolute (pimenta officinalis myrtaceae), pimento berry oil (pimento officinalis myrtaceae), pimento officinalis myrtaceae berry oil, pimento oil (pimenta officinalis lindl.), pimpinella anisum l. seed oil, pinang (areca catechu linn.), pine mountain oil (pinus mugo turra var. pumilio), pine needle absolute (pinus pinaster ait.), pine needle oil (abies sibirica ledeb.) siberia, pine needle oil dwarf (pinus mugo turra var. pumilio), pine norway oil (pinus sylvestris), pine oil scotch (pinus sylvestris), pine tar oil (pinus spp.), pink frangipanni absolute (plumiera rubra), pinus mugo turra var. pumilio (haenke) zenari oil, pinus pinaster ait absolute, pinus spp. oil, pinus sylvestris l. oil, piper betel leaf oil, piper cubeba l. oil, piper cubeba l. oleoresin, piper nigrum black absolute, piper nigrum l. oil black india, piper nigrum l. oil white, piper nigrum l. oleoresin black, piper nigrum l. oleoresin white, pistacia lentiscus absolute, pistacia lentiscus oil, pistacia lentiscus resin, pistacia lentiscus resinoid, plumiera rubra absolute, pogostemon patchouli absolute, pogostemon patchouli oil, polianthes tuberosa l. absolute pommade, polianthes tuberosa l. oil, poplar bud oleoresin, port orford cedarwood oil (chamaecyparis lawsoniana), prickly ash bark (angelica tree), prickly ash bark (xanthoxylum americanum), prickly ash berries, prickly ash berries (angelica tree), prickly elder bark, prickly elder berries, pride of china (melia azadirachta), protium gileadense, prunus amygdalus amara l. oil, prunus armeniaca l., prunus laurocerasus l. oil, pseudotsuga taxifolia, pterocarpus santalinus oil, pulsatilla nuttaliane, pyrus americana d.c., red alder bark (alnus seruulata willd.), red cedarwood oil (juniperus virgniana l.) america, red cockscomb (amaranthus hypochondriacus linn.), red pepper oleoresin, red sandalwood oil (pterocarpus santalinus), red spruce oil (picea rubens sarg.), reseda absolute pomade, ribes nigrum bud absolute, ribes nigrum l. bud oil, rosa centifolia absolute morocco, rosa centifolia leaf absolute, rosa centifolia oil morocco, rosa darnascena mill. absolute bulgaria, rosa damascena mill. oil bulgaria, rose absolute (rosa damascena mill.) bulgaria, rose de mai absolute (rosa centifolia) morocco, rose geranium absolute (pelargonium graveolens), rose geranium oil (pelargonium graveolens) bourbon, rose geranium oil terpeneless (pelargonium graveolens), rose leaf absolute (rosa centifolia), rose oil (rosa centifolia) morocco, rose oil (rosa damascena mill.) bulgaria, rose oil (rosa damascena mill.) turkey, rose oil otto (rosa damascena mill.) bulgaria, rose wood oil (aniba rosaeodora var. amazonia ducke) brazil, rosemary absolute (rosmarinus officinalis), rosemary oil (rosmarinus officinalis) morocco, rosemary oil (rosmarinus officinalis) spain, rosemary oleoresin (rosmarinus officinalis), rosemary terpeneless (rosmarinus officinalis), rosmarinus officinalis l. absolute, rosmarinus officinalis l. oil morocco, rosmarinus officinalis l. oil spain, rowan tree (pyrus aucuparia gaertn), royal staff, rue oil (ruta graveolens), ruta graveolens l. oil, saffron oil (crocus sativus), saffron resinoid (crocus sativus), sage oil (salvia lavandulaefolia spp. vellerea) spain, sage oil (salvia lavandulaefolia vahl.) spain, sage oil dalmatian (salvia officinalis), sage oleoresin dalmatian (salvia officinalis), sage oleoresin spanish (salvia lavandulaefolia vahl.), salt rheum weed (chelone glabra), salvia lavandulaefolia spp. vellerea oil spain, salvia lavandulaefolia vahl. oil spain, salvia officinalis l. oil, salvia sclarea l. absolute, salvia sclarea l. oil france, salvia sclarea l. resin america, sandalwood east indian oil (santalum album), sandalwood oil (santalum cygnorum) australia, sandalwood oil west indian (amyris balsamifera), sandalwood oil yellow (santalum album), sandalwood resinoid, sandalwood yellow chips, santalum album l. oil, santalum cygnorum l. oil australia, sassafras officinale oil, sassafras oil (sassafras officinale), satureja hortensis l. oil, satureja hortensis l. oleoresin, satureja montana l. oil, satureja montana l. oleoresin, saussurea lappa clarke absolute, saussurea lappa clarke resinoid, saussurea lappa clarke root oil, savin oil (juniperus phoenicea), savin oil (juniperus sabina), savory summer oil (satureja hortensis l.), savory summer oleoresin (satureja hortensis l.), savory winter oil (satureja montana l.), savory winter oleoresin (satureja montana), schinus molle l. berry oil, schinus molle l. leaf oil, scolymus cardunculus, scotch pine oil (pinus sylvestris), sea beet, seaweed absolute (fucus vesiculosus et serratus), senegal gum (acacia senegal l. willd), senegambia (andira inermis), serpent's tongue (erythronium americanum ker-gawl), shellflower (chelone glabra), shiu leaf oil (cinnamomum camphora l. nees se eberm.), shot bush (aralia nudicaulis), shrubby trefoil, silkweed (asclepias syriaca willd.), silver fir oil (abies balsamea l. mill.) america, silver leaf (gnaphalium polycephalum), silver spruce oil from needles (abies alba mill), sitka spruce oil, smallage herb oil (levisticum officinale koch), smallage oleoresin (levisticum officinale koch), smallage root oil (levisticum officinale koch), smell fox (anemone nemorosa linn.), smellage root oil (levisticum officinale koch), smooth alder bark (alnus serrulata willd.), snake head (chelone glabra), snake root oil (asarum canadense) canada, south american spp. of copaifera l. oil, spanish bugloss root (alkanna tinctoria tausch.), spanish needles (bidens bipinnata linn.), spartium junceum l. absolute, spearmint absolute (mentha spicata), spearmint oil (mentha spicata), spearmint oil terpeneless (mentha spicata), spike lavender oil (lavandula latifolia v.l.), spikenard oil (nardostachys jatamansi), spilanthes acmelia oleracea oleoresin, spinach beet, spotted gum oil (eucalyptus citriodora), spreading orache (atriplex patuia), spruce needle absolute, spruce oil (tsuga canadensis), spruce oil black (picea mariana mill.), spruce oil red (picea rubens sarg.), spruce silver oil from cones (abies alba mill), spruce sitka oil (picea sitchensis), spruce white oil from cones (abies alba mill), st. john's herb (eupatorium cannabinum linn.), stachys sieboldii, star anise (illicium verum hook f.), star anise oil (illicium verum hook f.) china, star anise oil terpeneless (illicium verum hook f.), star anise seed (illicium verum hook f.), stinking arrach, stinking goosefoot, stinking motherwort, storax oil, storax resin, storax resinoid (liquidamber spp.), strawberry blite (amaranthum blitum linn.), strawberry tree (arbutus unede), styrax benzoin dry and sumatra absolute, styrax oil, styrax resin, styrax resinoid (liquidamber spp.), styrax tonkinensis craib ex hartwiss absolute, sugandha kokila berry oil, sugi wood oil (cryptomeria japonica d. don.), summer savory oil (satureja hortensis), sunflower artichoke (helianthus tuberosus), surfleurs d'oranger oil, swamp dogwood, sweatroot (polemonium reptans linn.), sweet almonds (amygdalus communis linn. var. dulcis), sweet balm leaves (melissa officinalis), sweet pea absolute (lathyrus latifolius), sweet scented life everlasting (gnaphalium polycephalum), symphytum officinalis extract, syringa vulgaris blossom extract, syringa vulgaris l. absolute, syringa vulgaris leaf extract, tag alder bark (alnus serrulata willd.), tagete oil (tagetes glandulifera l.) mexico, tagete oil (tagetes minuta l.) egypt, tagetes glandulifera l. oil mexico, tagetes minuta l. absolute egypt, tagetes minuta l. oil egypt, tagetes oil (tagetes patula l.) india, tagetes patula l. absolute india, tallow shrub (myrica cerifera), tanacetum vulgara extract, tanacetum vulgara l. oil, tangerine oil (citrus reticulata blanco) dancy, tangerine oil terpeneless (citrus reticulata blanco), tansy extract (tanacetum vulgara), tansy oil (tanacetum vulgara), taraxacum officinale extract, tarragon extract (artemisia dracunculus), tarragon oil (artemisia dracunculus), tarragon oleoresin, tasmamia lanceolata oil, tea tree extract (melaleuca alternifolia), tea tree oil (melaleuca alternifolia) australia, the hummingbird tree (chelone glabra), thuja extract (thuja occidentalis), thuja occidentalis extract, thuja occidentalis l. oil canada, thuja plicata oil, thujopsis dolabrata l. wood oil, thyme absolute (thymus vulgaris), thyme extract (thymus vulgaris), thyme extract wild or creeping (thymus serpyllum), thyme oil (thymus zygis spp. gracillis) spain, thyme oil (thymus zygis spp. sylvestris) spain, thyme oil red (thymus vulgaris) india, thyme oil red (thymus vulgaris) spain, thyme oil white (carum copticum benth. et hook), thyme oil wild or creeping (thymus serpyllum), thyme oleoresin, thymus capitatus l. hoffmanns & link oil spain, thymus hiemalis l. absolute spain, thymus hiemalis l. oil spain, thymus mastichina l. oil spain, thymus serpyllum l. extract, thymus serpyllum l. oil, thymus vulgaris absolute, thymus vulgaris extract, thymus vulgaris oil india, thymus vulgaris oil spain, thymus zygis spp. gracillis oil spain, thymus zygis spp. sylvestris oil spain, tilam wangi oil, tilia cordata flower extract, ti-trol (melaleuca alternifolia) australia, toadroot (actaea spicata), tobacco flower absolute (nicotiana spp.), tobacco leaf absolute (nicotiana spp.), tolu balsam (myroxylon toluiferum), tolu balsam absolute (myroxylon balsanum), tolu balsam extract (myroxylon toluiferum), tolu balsam oil (myroxylon toluiferum), tolu balsam resin (myroxylon toluiferum), tolu balsam resinoid (myroxylom balsanum), tolutanischer balsam (myroxylon toluiferum), tonka bean absolute (dipteryx odorata), tonka bean oleoresin (dipteryx odorata), tonka bean resinoid (dipteryx odorata), toothache tree bark (angelica tree), toothache tree bark (xanthoxylum americanum), toothache tree berries (angelica tree), trachyspermum ammi seed oil, treemoss absolute (evernia furuaceae), treemoss resinoid (evernia furfaceae spp.), trigonella foenum graecum extract, trigonella foenum graecum l. absolute, trigonella foenum graecum l. oleoresin, tsuga canadensis l. oil, tuberose absolute pommade (polianthes tuberosa), tuberose oil (polianthes tuberosa), turkish geranium oil (cymbopogon martini roxb. stapf) india, turmeric oleoresin (curcuma longa), turmeric root oil (curcuma longa), turmeric root oleoresin (curcuma longa), turtle bloom (chelone glabra), turtle head (chelone glabra), valerian root extract (valeriana officinalis), valerian root oil (valeriana officinalis), valeriana officinalis l. root oil, valeriana officinalis root extract, vanilla absolute (vanilla spp.) 100%, vanilla aromatica extract, vanilla bean extract (vanilla spp.), vanilla oleoresin (vanilla spp.) bali, vanilla resinoid (vanilla spp.), vanilla spp. oleoresin bali, velvet flower (amaranthus hypochondriacus linn.), verbena absolute (lippia citriodora) france, verbena absolute spanish (thymus hiemalis) spain, verbena extract (verbena officinalis), verbena officinalis extract, verbena oil (lippia citriodora cymbopogon spp.) france, verbena oil spanish (thymus hiemalis) spain, verbena oil terpeneless, vervain extract (verbena officinalis), vetiver oil (vetiveria zizaniodes stapf.) haiti, vetiver resinoid (vetiveria zizaniodes stapf.), vetiveria zizaniodes stapf. oil haiti, viola odorata extract, viola odorata l. flower absolute, viola odorata l. leaf absolute, violet extract (viola odorata), violet flower absolute (viola odorata), violet leaf absolute (viola odorata), viscum album absolute, voucapoua araroba, wachsgagle (myrica cerifera), wafer ash, wapatoo, water agrimony (bidens tripartita linn.), water avens (geum rivale linn.), water flower (geum rivale linn.), way bennet (geum urbanum linn.), weeping ash (fraxinus excelsior), west indian rosewood oil (amyris balsamifera), west indian sandalwood oil (amyris balsamifera), western red cedarleaf oil (thuja plicata donn ex. d. don), white asphodel (asphodelus ramosus), white beet (mangel wurzel), white chelone (chelone glabra), white cinnamon bark oil (canella alba), white flag extract, white lily extract (lilium candidum), white pepper oil (piper nigrum), white pepper oleoresin (piper nigrum), wild amaranth (amaranthum blitum linn.), wild apple, wild apple (malus communis), wild arrach (atriplex patuia), wild basil (calamintha clinopodium), wild ginger oil (asarum canadense) canada, wild liquorice (aralia nudicaulis), wild nard (asarum europaeum), wild rye (geum urbanum linn.), wild sarsaparilla (aralia nudicaulis), wine lie oil white, winter pink (epigaea repens linn), wintergreen oil (gaultheria procumbens) china, wood anemone (anemone nemorosa linn.), worm bark (andira inermis), wormseed oil (chenopodium ambrosioldes var. anthelminthicum) america, wormwood oil (artemisia absinthium america, wormwood oil annus (artemisia annua), yarrow herb (achillea millefolium), yarrow oil (achillea millefolium), yellow bedstraw (galium verum), yellow leaf cup (polymnia uvedalia), yellow snowdrop (erythronium americanum ker-gawl) america, yellow wood bark (xanthoxylum americanum), ylang ylang absolute (cananga odorata hook. f. and thomas.), ylang ylang extract (cananga odorata hook. f. and thomas.), ylang ylang oil (cananga odorata hook. f. and thomas.), yomugi oil (artemisia vulgaris), zingiber officinale rosc. absolute, zingiber officinale rosc. oleoresin, zingiber officinale rosc. oleoresin africa, zingiber officinale rosc. root extract, zingiber officinale rosc. root oil cochin and zvoulimba oil (eucalyptus dives).

In some embodiments it is advantageous to add additional components to a composition of the present invention.

One additional component useful in implementing a composition of the present invention is benzyl benzoate. Benzyl benzoate (benzoic acid benzyl ester, CAS nr. 120-51-4) is useable in implementing a composition of the present invention. Preferably, the amount of benzyl benzoate in the composition, when present, is between about 0.1% and up to 60% by weight of the composition.

Another additional component optionally useful in implementing a composition of the present invention is amyl salicylate. Generally iso (3-methylbutyl ester) and normal (pentyl ester) isomers are used alone or in combination in an composition of the present invention. Preferably, the total amount of amyl salicylate (iso and normal) in the composition, when present, is between about 0.1% and 55% by weight of the composition.

Another additional component optionally useful in implementing a composition of the present invention is an aldehyde component. Generally, one aldehyde or a mixture of aldehydes is used as an additional component, the aldehydes preferably having a general structure RCH═O, wherein R is selected from the group consisting of aryl and alkyl groups, preferably alkyl groups (branched or linear) where R is between 8 and 16 carbon atoms. Especially preferred is a mixture including one or more aldehydes from the commonly available C8 to C₁₋₆ aldehydes: aldehyde C8 (Octanal, CAS nr. 124-13-0), aldehyde C9 (Nonanal, CAS nr. 124-19-6), aldehyde C10 (Decanal, CAS nr. 112-31-2), aldehyde C11 (Undecanal, CAS nr. 112-44-7), aldehyde C12 (lauryl aldehyde, CAS nr. 112-54-9), aldehyde C13/C15 (mixture of isomers, CAS nr. 9382-14-3), aldehyde C14 (myristaldehyde, CAS nr. 124-25-4) and aldehyde C16 (mixture of isomers, CAS nr. 77-83-8). Preferably, the total amount of aldehyde component in a composition of the present invention, when present, is between about 0.1% and 55% by weight of the composition.

In some embodiments of the present invention it is advantageous to add an auxiliary component to a composition of the present invention, the auxiliary component having a discernable smell. Suitable auxiliary components are generally (but not necessarily) known in the art of aromatherapy. Auxiliary compounds are compounds that may be but are not necessarily involved in affecting receptors for modifying perceptions of a malodor but often have a desired aromatherapic effect. Effects evoked by smelling various compounds are well known to one skilled in the art of aromatherapy. An additional use of an auxiliary component is as an indicator: as long as the auxiliary component is smelled, the composition is still effective. Preferred auxiliary components are vanillin and ethyl vanillin, both having a pleasant smell and known to one skilled in the art as having a calming effect on human beings.

In embodiments of the present invention, a composition of the present invention comprises at least one auxiliary component selected from the group consisting of vanillin, ethyl vanillin, extract of Hedychium, extract of Menta, extract of Citrus and mixtures thereof. Preferably, the total amount of auxiliary component in a composition of the present invention, when present, is up to about 60% by weight of the composition.

Ethyl vanillin (4-hydroxy-3-ethoxybenzaldehyde, CAS nr. 121-32-4) is usable as an auxiliary component in a composition of the present invention.

Vanillin (4-hydroxy-3-methoxybenzaldehyde, CAS nr. 121-33-5) is usable as an auxiliary component in a composition of the present invention.

Extract of Hedychium (preferably Hedychium spicatum) is usable as an auxiliary component in a composition of the present invention. Preferred is the extract of the root of Hedychium, preferably an essential oil of the root of Hedychium, especially the essential oil of the root of Hedychium spicatum.

Extract of Menta (preferably Menta piperata) is usable as an auxiliary component in a composition of the present invention. Preferred is an oil of Menta, preferably an essential oil of Menta, especially the essential oil of Menta piperata.

Extract of Citrus (especially Citrus Limonum) is usable as an auxiliary component in a composition of the present invention. Preferred is an oil of Citrus, especially an essential oil of Citrus, especially the essential oil of Citrus Limonuin.

Further substances useful as additional components or as auxiliary components useful in implementing the teachings of the present invention include but are not limited to: acetal, acetaldehyde diethyl acetal, acetaldehyde dimethyl acetal, acetaldehyde dipropyl acetal, acetaldehyde ethyl (z)-3-hexen-1-yl acetal, acetaldehyde ethyl phenethyl acetal, acetaldehyde phenethyl propyl acetal, acetanisole, acetate c-10, acetate c-11, acetate c-12, acetate c-6, acetate c-7, acetate c-8, acetate c-9, acetic acid, acetoacetic ester, acetoin, acetophenone, acetyl eugenol, acetyl isoeugenol, acetyl methyl carbinol, acetyl vanillin, alcohol c-10, alcohol c-11 undecylenic, alcohol c-11 undecylic, alcohol c-12, alcohol c-7, alcohol c-8, alcohol c-9, aldehyde c-10, aldehyde c-10 dimethyl acetal, aldehyde c-11 moa, aldehyde c-11 undecylenic, aldehyde c-11 undecylic, aldehyde c-12 lauric, aldehyde c-12 nma, aldehyde c-14, aldehyde c-14 myristic, aldehyde c-16, aldehyde c-18, aldehyde c-19, aldehyde c-6, aldehyde c-7, aldehyde c-8, aldehyde c-9, allyl 3-phenyl-2-propenoate, allyl α-ionone, allyl amyl glycolate, allyl caproate, allyl capronate, allyl cinnamate, allyl cyclocitrylidene acetone, allyl cyclohexane propionate, allyl cyclohexyl acetate, allyl cyclohexyl propionate, allyl hexanoate, allyl phenoxyacetate, ambrette gx, ambrettolide, ambrox, ambroxan, ambroxide, amyl caproate, amyl cinnamyl acetate, amyl ethyl carbinol, amyl hexanoate, amyl iso formate, amyl iso octanoate, amyl vinyl ketone, angelica lactone, anhydrolinalool oxide, anisic aldehyde, anisyl acetate, anisyl acetone, anisyl alcohol, anisyl formate, anisyl methyl ketone, benzaldehyde, benzaldehyde dimethyl acetal, benzophenone, benzyl 2-methyl propionate, benzyl 3-phenyl propenoate, benzyl acetate, benzyl alcohol, benzyl benzoate, benzyl butyrate, benzyl carbinyl 2-methyl butyrate, benzyl carbinyl 3-methyl butanoate, benzyl carbinyl acetate, benzyl carbinyl benzoate, benzyl carbinyl butyrate, benzyl carbinyl cinnamate, benzyl carbinyl formate, benzyl carbinyl isobutyrate, benzyl carbinyl phenyl acetate, benzyl carbinyl propionate, benzyl carbinyl salicylate, benzyl cinnamate, benzyl dimethyl carbinyl acetate, benzyl dimethyl carbinyl butyrate, benzyl formate, benzyl isoamyl ether, benzyl isobutyrate, benzyl isoeugenol, benzyl ortho-hydroxybenzoate, benzyl phenyl acetate, benzyl phenyl formate, benzyl propionate, benzyl salicylate, benzyl tiglate, benzyl trans-2-methyl-2-butenoate, bois de rose oxide, butyl (s)-(−)-lactate, butyl 2-methyl butyrate, butyl 2-methyl propanoate, butyl acetate, butyl benzoate, butyl butyrate, butyl butyryl lactate, butyl cinnamate, butyl heptanoate, butyl heptoate, butyl isobutyrate, butyl laevo-lactate, butyl pentanoate, butyl phenyl acetate, butyl propenyl ketone, butyl propionate, butyl valerate, butyric acid, camphene, campholenic aldehyde, camphor gum, caramel furanone, carvyl acetate, carvyl propionate, cedr-8-enyl methyl ketone, cedran-8-yl acetate, cedrenol, cedrol, cedryl acetate, cedryl methyl ether, cinnamaldehyde, cinnamic aldehyde, cinnamyl acetate, cinnamyl alcohol, cinnamyl butyrate, cinnamyl cinnamate, cinnamyl formate, cinnamyl isobutyrate, cinnamyl isovalerate, cinnamyl propionate, citral diethyl acetal, citral, cis, trans, citronellal, citronellol, citronellyl acetate, citronellyl butyrate, citronellyl formate, citronellyl isobutyrate, citronellyl nitrile, citronellyl propionate, citronellyl valerate, coumarin, creosol, cuminaldehyde, cyclamen aldehyde, cyclohexapyrazine, cyclohexyl butyrate, cyclohexyl isobutyrate, dec-9-en-1-ol, decanal, decanal dimethyl acetal, decanoic acid, decyl acetate, decyl alcohol, decyl butyrate, decyl propionate, decylic acid, dehydro-β-cyclocitral, dehydroxyliaalool oxide, diacetyl, diasymol acetate, dibenzyl ketone, diethyl malonate, difurfuryl disulfide, dihydroanethole, dihydrocarveol, dihydrocarvyl acetate, dihydrocinnamic acetate, dihydrocinnamyl alcohol, dihydrocoumarin, dihydroeugenol, dihydrojasmone, dihydrojasmone lactone, dihydrolinalool, dihydromyrcenol, dihydromyrcenyl acetate, dimethoxyphenyl methane, dimethyl acetal, dimethyl anthranilate, dimethyl benzyl carbinol, dimethyl benzyl carbinyl acetate, dimethyl benzyl carbinyl butyrate, dimethyl benzyl carbinyl formate, dimethyl dihydrocyclopentapyrazine, dimethyl diketone, dimethyl octanol, dimethyl octanyl acetate, dimethyl phenyl ethyl carbinol, diphenyl ether, diphenyl oxide, dipropylene glycol, estragole, ethyl (e)-2-hexenoate, ethyl (e)-2-octenoate, ethyl 10-undecenoate, ethyl 2,4-hexadienoate, ethyl 2-hydroxybenzoate, ethyl 2-hydroxypropionate, ethyl 2-methyl butanoate, ethyl 2-methyl butyrate, ethyl 2-methyl propanoate, ethyl 3-(methyl thio)propionate, ethyl 3-hydroxybutanoate, ethyl 3-methyl butanoate, ethyl 3-phenyl propenoate, ethyl 4-oxopentanoate, ethyl acetate, ethyl acetoacetate, ethyl benzoate, ethyl butyl ketone, ethyl butyrate, ethyl caprate, ethyl caproate, ethyl caprylate, ethyl cinnarate, ethyl citrate, ethyl decanoate, ethyl dodecylate, ethyl formate, ethyl heptanoate, ethyl heptoate, ethyl hexanoate, ethyl isobutyrate, ethyl isovalerate, ethyl lactate, ethyl laurate, ethyl levulinate, ethyl malonate, ethyl methyl mercaptopropionate, ethyl methyl phenyl glycidate, ethyl myristate, ethyl nonanoate, ethyl octanoate, ethyl oxyhydrate, ethyl pelargonate, ethyl phenyl acetate, ethyl propionate, ethyl salicylate, ethyl sorbate, ethyl tetradecanoate, ethyl tiglate, ethyl trans-2-hexenoate, ethyl trans-2-methyl-2-butenoate, ethyl trans-2-octenoate, ethyl undecanoate, ethyl undecylate, ethyl undecylenate, ethyl vanillin, ethyl vinyl carbinol, ethyl-3-hydroxybutyrate, eucalyptol, eugenol, eugenyl acetate, eugenyl methyl ether, farnesene, farnesyl acetone, furaneol, furfual, furfuryl acetate, furfryl alcohol, furfuryl mercaptan, furfuryl valerate, geosmin, geranic oxide, geraniol, geranyl acetate, geranyl acetone, geranyl butyrate, geranyl formate, geranyl isobutyrate, geranyl propionate, glyceryl triacetate, guaiacol, guaiacyl phenyl acetate, hedione, heliotropin, heliotropin acetate, heliotropyl acetate, heptanal, heptyl acetate, heptyl alcohol, heptylidene acetone, hexanal, hexyl 3-methyl butanoate, hexyl acetate, hexyl benzoate, hexyl butyrate, hexyl caproate, hexyl caprylate, hexyl hexanoate, hexyl isobutyrate, hexyl isovalerate, hexyl octanoate, hexyl phenyl acetate, hexyl propionate, hexyl tiglate, hexyl trans-2-methyl-2-butenoate, hyacinth body, hyacinthin, hydratropaldehyde, hydratropaldehyde dimethyl acetal, hydratropic acetate, hydratropic aldehyde, hydratropic aldehyde dimethyl acetal, hydratropyl acetate, hydrocinnamaldehyde, hydrocinnamyl cinnamate, hydrocinnamyl propionate, hydroxycitronellal, hydroxycitronellal diethyl acetal, hydroxycitronellal dimethyl acetal, hydroxycitronellol, indole, lactic acid butyl ester butyrate, lauryl acetate, lauryl alcohol, lauryl aldehyde, leerall, ligusticum lactone, lilyall, linalool, linalool oxide, linalyl acetate, linalyl benzoate, linalyl butyrate, linalyl isobutyrate, linalyl propionate, longifolene, maltol, melonal, menthalactone, methyl (e)-2-octenoate, methyl (e)-3-phenyl propenoate, methyl (e)-cinnamate, methyl 2-furan carboxylate, methyl 2-furoate, methyl 2-hydroxybenzoate, methyl 2-methyl butanoate, methyl 2-methyl butyrate, methyl 2-nonenoate, methyl 2-nonynoate, methyl 2-octynoate, methyl 2-pent-2-enyl-3-oxo-1-cyclopentyl acetate, methyl 2-pyrrolyl ketone, methyl 2-thiazolyl ketone, methyl 5-methyl-2-furyl ketone, methyl α-toluate, methyl anthranilate, methyl atratate, methyl benzoate, methyl caproate, methyl caprylate, methyl cedryl ketone, methyl chavicol, methyl cyclopentenolone, methyl decalactone, methyl dihydrojasmonate, methyl eugenol, methyl heptenone, methyl heptin carbonate, methyl heptyl carbinol, methyl hexanoate, methyl isobutyl ketone, methyl isoeugenol, methyl jasmonate, methyl octanoate, methyl octenoate, methyl octine carbonate, methyl para-propyl phenyl ether, methyl para-tolyl ketone, methyl pentanol, methyl pentenyl ketone, methyl phenyl acetate, methyl phenyl ethyl ether, methylpyrazinyl ketone, methyl salicylate, methyl undecyl ketone, methyl undecylenate, methyl vanillin, methyl-2-pyridyl ketone, methyl-α-naphthyl ketone, mint lactone, moschus lactone, mushroom alcohol, musk gx, musk ketone, musk xylol, myrcene, myristaldehyde, myrmac aldehyde, myrtenol, myrtenyl acetate, neofolione, nerol, nerol oxide, nerolin bromelia, neryl acetate, neryl isobutyrate, nonanal, nonyl acetate, nonyl alcohol, nonylene glycol diacetate, nopol, nopyl acetate, ocimene, ocimene quintoxide, oct-1-en-3-yl acetate, octaldehyde dimethyl acetal, octanal, octanal dimethyl acetal, octen-1-yl acetate, octyl 2-methyl propanoate, octyl acetate, octyl alcohol, octyl butyrate, octyl formate, octyl isobutyrate, oil of niobe, oleic acid, oranger crystals, oranger liquid, pentyl 2-furyl ketone, perilla alcohol, phantolid, phenethyl 2-furoate, phenethyl 2-methyl butyrate, phenethyl acetate, phenethyl alcohol, phenethyl benzoate, phenethyl butyrate, phenethyl cinnamate, phenethyl formate, phenethyl hexanoate, phenethyl isobutyrate, phenethyl isovalerate, phenethyl methyl ether, phenethyl phenyl acetate, phenethyl pivalate, phenethyl propionate, phenethyl salicylate, phenethyl tiglate, phenol carbinol, phenyl acetaldehyde, phenyl acetaldehyde dimethyl acetal, phenyl allyl cinnamate, phenyl methyl 3-phenyl-2-propenoate, phenyl methyl acetate, phenyl methyl ketone, phenyl methyl propanoate, phenyl propyl acetate, phenyl propyl alcohol, phenyl propyl aldehyde, phenyl propyl cinnamate, pin-2-en-4-one, piperonal, prenol, prenyl benzoate, propyl acetal, propyl butyrate, propyl formate, propyl iso methanoate, propyl propanoate, propyl propionate, protocatechuic aldehyde ethyl ether, raspberry ketone, raspberry ketone methyl ether, rhodinol, rhodinyl acetate, rhodinyl isobutyrate, rum ether, safranal, salicylaldehyde, santall, sclareolide, spiroxide, strawberry furanone, styralyl acetate, styralyl propionate, sulfurol, syringaldehyde, syringol, terpinen-4-ol, terpinolene, terpinyl acetate, terpinyl propionate, tetrahydro-6-nonyl-2h-pyran-2-one, tetrahydroalloocimenol, tetrahydrogeraniol, tetrahydrogeranyl acetate, tetrahydrolinalool, tetrahydromuguol, tetrahydromyrcenol, theaspirane, thymol, tiglaldehyde, tiglic aldehyde, triacetin, tricyclodecenyl acetate, triethyl citrate, trimethyl cyclopentenyl acetaldehyde, trimethylpyrazine, turberyl acetate, undecanal, undecanoic acid, undecylic acid, valencene, valspice, vanillaldehyde, vanillin, vanillin acetate, vanillyl acetone, vanillyl alcohol, veratraldehyde, verbenol, verdyl acetate, verymoss, vetiverol, vinegar naphtha, vinyl sulfurol, yara yara, zingerone, (−)-α-bisabolol, (−)-cis-rose oxide, (−)-iso borneol, (+)-bornan-2-one, (+)-nootkatone, (+)-β-pinene, (+/−)-3,7-dimethyl-6-octen-1-ol, (+/−)-α-citronellol, (1r)-(−)-myrtenal, (1r)-(+)-fenchyl alcohol, (1s)-(−)-verbenone, (2e, 6z)-dodeca-2, (e)-cinnamic acid, (e)-cinnamyl nitrile, (e)-decenaldehyde, (e)-2-decen-1-al, (e)-2-decenoic acid, (e)-2-hepten-1-al, (e)-2-hexen-1-al, (e)-2-hexen-1-ol, (e)-2-hexen-1-yl acetate, (e)-2-hexen-1-yl butanoate, (e)-2-hexen-1-yl butyrate, (e)-2-methyl-2-buten-1-al, (e)-2-methyl-2-pentenoic acid, (e)-2-nonen-1-al, (e)-2-nonen-1-ol, (e)-2-octen-1-al, (e)-2-octen-1-yl acetate, (e)-2-penten-1-al, (e)-2-tridecen-1-al, (e)-2-(e)-4-decadien-1-al, (e)-2, (z)-6-nonadien-1-al, (e)-2, (z)-6-nonadien-1-ol, (e)-2, (e)-4-heptadien-1-al, (e)-2, (e)-4-hexadien-1-al, (e)-2, (e)-4-undecadien-2,4-al, (e)-2, (z)-6-dodecadien-1-al, (e)-2-undecen-1-al, (e)-α-damascone, (e)-α-ionone, (e)-α-damascone, (e, e)-2,4-undecadien-1-al, (e/z)-ethyl 3-phenyl oxirane-2-carboxylate, (e/z)-ethyl phenyl glycidate, (r)-(−)-carvone, (s)-(−)-dihydrocuminic alcohol, (z)-jasmone, (z)-rose oxide, (z)-1-methyl-4-isopropyl-3-cyclohexanone, (z)-2-nonen-1-ol, (z)-3-hexen-1-al, (z)-3-hexen-1-ol, (z)-3-hexen-1-yl (e)-2-methyl 2-butenoate, (z)-3-hexen-1-yl 2-hydroxypropanoate, (z)-3-hexen-1-yl 2-methyl butanoate, (z)-3-hexen-1-yl 2-methyl butyrate, (z)-3-hexen-1-yl 2-methyl propionate, (z)-3-hexen-1-yl 3-methyl butanoate, (z)-3-hexen-1-yl acetate, (z)-3-hexen-1-yl benzoate, (z)-3-hexen-1-yl butanoate, (z)-3-hexen-1-yl butyrate, (z)-3-hexen-1-yl caproate, (z)-3-hexen-1-yl ethanoate, (z)-3-hexen-1-yl ethyl acetal of acetaldehyde, (z)-3-hexen-1-yl formate, (z)-3-hexen-1-yl hexanoate, (z)-3-hexen-1-yl isobutyrate, (z)-3-hexen-1-yl isovalerate, (z)-3-hexen-1-yl lactate, (z)-3-hexen-1-yl methanoate, (z)-3-hexen-1-yl pentanoate, (z)-3-hexen-1-yl phenyl acetate, (z)-3-hexen-1-yl propionate, (z)-3-hexen-1-yl tiglate, (z)-3-hexen-1-yl valerate, (z)-4-decen-1-al, (z)-4-hepten-1-ol, (z)-4-hexen-1-ol, (z)-5-octen-1-ol, (z)-6-nonen-1-al, (z)-6-nonen-1-ol, (z)-7-decen-1-al, (z)-ortho-coumarinic acid lactone, (z/e)-acroval, (z/e)-amyl cinnamic aldehyde, (z/e)-citral dimethyl acetal, (z/e)-furfural acetone, (z/e)-furfurylidene acetone, (z/e)-2,4-dimethyl-3-cyclohexene carboxaldehyde, (z/e)-4-formyl-1,3-dimethyl cyclohex-1-ene, (z/e)-α-amyl cinnamaldehyde, 1-ethyl hexyl acetate, 1-formyl-4-isohexenyl-4-cyclohexene, 1-hexen-3-ol, 1-hexen-3-yl acetate, 1-methyl-4-(1-methyl ethyl)-7-oxabicyclo(2.2.1)heptane, 1-methyl-4-(4-methyl-3-pentenyl)-3-cyclohexen-1-carboxaldehyde, 1-octanol, 1-octen-3-ol, 1-octen-3-one, 1-penten-3-ol, 1-phenoxy-1-propoxyethane, 1-vinyl butan-1-ol, 1-(3-methylpyrazinyl)ethan-1-one, 1,4-cineole, 1,8-cineole, 1,3-nonanediol acetate, 1,3,5-undecatriene, 10-undecen-1-al, 10-undecen-1-ylacetate, 12-oxa hexadecanolide, 16-hydroxy-12-oxahexadecanoic-omega-lactone, 1-para-hydroxyphenyl-3-butanone, 1-para-menthen-8-ol, 1-undecanol, 2-acetyl furan, 2-acetyl pyrazine, 2-acetyl pyridine, 2-acetyl pyrrole, 2-acetyl thiazole, 2-acetyl-3-methylpyrazine, 2-acetyl-5-methyl furan, 2-camphanyl acetate, 2-ethyl furan, 2-ethyl hexanal cycoglycol acetal, 2-ethyl oxole, 2-ethyl-3, 5{6}-dimethyl pyrazine, 2-ethyl-3-methylpyrazine, 2-ethyl-4-butanol, 2-ethyl-4-methyl thiazole, 2-furan carbinol, 2-furyl disulfide, 2-furyl methane thiol, 2-furyl methyl ketone, 2-furyl pentyl ketone, 2-hexen-4-one, 2-methoxy-3-(1-methyl propyl)pyrazine, 2-methoxy-3-isobutyl pyrazine, 2-methoxy-4-allyl phenol, 2-methoxy-4-ethyl phenol, 2-methoxy-4-propenyl phenol, 2-methyl 2-methyl propanoate, 2-methyl aminomethyl benzoate, 2-methyl butane-1-carboxylic acid, 2-methyl butyl 2-methyl butanoate, 2-methyl butyl 2-methyl butyrate, 2-methyl butyl acetate, 2-methyl decan-1-al, 2-methyl propyl benzoate, 2-methyl propyl phenyl acetate, 2-methylpyrazine, 2-methyl undecanal, 2-methyl-1-propyl acetate, 2-methyl-2-penten-1-al, 2-methyl-3-phenyl-2-propen-1-al, 2-nonanol, 2-octen-4-one, 2-pentyl furan, 2-pentyl-3-methyl-2-cyclopenten-1-one, 2-phenyl ethanol, 2-phenyl ethyl caproate, 2-phenyl ethyl trans-2-methyl butenoate, 2-phenyl propionaldehyde, 2-phenyl-5-methyl-2-hexen-1-al, 2-propen-1-yl cyclohexane acetate, 2-propenyl 2-((3-methyl butyl)oxy)ethanoate, 2-propyl iso-3,5 or 6-methoxypyrazine, 2-tridecanone, 2-(phenyl methylene)octanal, 2-(1-ethyl pentyl)-1,3-dioxolane, 2-(1-methyl propyl)thiazole, 2,3-diethyl pyrazine, 2,3-diethyl-5-methylpyrazine, 2,3-dimethyl-1,4-diazine, 2,4-dimethyl crotonaldehyde, 2,5-diethyl pyrazine, 2,6-dimethoxyphenol, 2,6-dimethylpyrazine, 2,2-dimethyl-3-methylene bicyclo(2.2.1)heptane, 2,3-benzopyrrole, 2,3,5,6-tetramethylpyrazine, 2,3-diethyl pyrazine, 2,4-octadien-1-al, 2,4,5-trimethyl thiazole, 2,5-dimethyl-1,4-diazine, 2-, 5{6}-methoxy-3-methylpyrazine, 2,6-dimethyl-5-hepten-1-al, 2,6-dimethyl-7-octen-2-ol, 2,6-dimethyl-7-octen-2-yl acetate, 2-iso butyl-3-methoxypyrazine, 2-isopropyl-3,5-methoxypyrazine, 2-sec-butyl thiazole, 2-sec-butyl-3(5/6)-methoxypyrazine, 3-acetopyridine, 3-acetyl pyridine, 3-allyl cyclohexanol propionate, 3-butyl phthalide, 3-butylidene phthalide, 3-decen-2-one, 3-ethyl-2-methylpyrazine, 3-heptanone, 3-hepten-2-one, 3-methyl butyl ethanoate, 3-methyl mercaptopropyl isothiocyanate, 3-methyl valeric acid, 3-methyl-2-buten-1-ol, 3-octanol, 3-octen-2-ol, 3-octyl acetate, 3-phenyl acrylic acid, 3-phenyl allyl alcohol, 3-phenyl propyl propionate, 3-phenyl-2-propen-1-yl 3-methyl butanoate, 3-phenyl-2-propen-1-yl propanoate, 3-propylidene phthalide, 3-(4-methyl cyclohex-3-en-1-yl)butyraldehyde, 3,7-dimethyl-6 & 7-octen-1-ol, 3,7-dimethyl-6 or 7-octen-1-yl acetate, 3,7-dimethyl-6 or 7-octen-1-yl isobutyrate, 3,7-dimethyl-6-octen-1-yl acetate, 3,7-dimethyl-6-octen-1-yl propionate, 3,7-dimethyl-6-octen-3-ol, 3,7-dimethyl-6-octene-1-nitrile, 3-ortho-methoxyphenyl-2-propenal, 4-carvomenthenol, 4-ethyl guaiacol, 4-ethyl-2-methoxyphenol, 4-hexen-3-one, 4-hydroxy-2,5-dimethyl-3(2h)-furanone, 4-methoxybenzyl alcohol, 4-methoxyphenyl acetone, 4′-methyl acetophenone, 4-methyl guaiacol, 4-methyl-1-isopropyl benzene, 4-methyl-2-pentanone, 4-methyl-2-phenyl-2-penten-1-al, 4-terpinenol, 4-(hydroxymethyl)-2-methoxyphenol, 4-(para-methoxyphenyl)-2-butanone, 4,5-dimethyl thiazole, 4,5-dimethyl-3-hydroxy-2,5-dihydrofuran-2-one, 4-oxoisophorone, 4-tert-butyl cyclohexanol, 4-tert-butyl cyclohexyl acetate, 5-acetyl-1,1,2,3,3,6-hexamethyl indan, 5-ethyl-4-hydroxy-2-methyl furan-3(2h)-one, 5-hydroxyethyl-4-methyl thiazole, 5-methyl furfural, 5-methyl-2-furaldehyde, 5-methyl-2-phenyl-2-hexen-1-al, 5,6,7,8-tetrahydroquinoxaline, 5h-5-methyl-6, 7-dihydrocyclopenta(β)pyrazine, 5-n-butyl-delta-valerolactone, 5-n-hexyl-5-hydroxypentanoic acid lactone, 5-tert-butyl-2,4,6-trinitro-metaxylene, 6-methyl quinoline, 6-methyl-5-hepten-2-one, 6,10,14-trimethyl-5,9,13-pentadecatrien-2-one, 8-cedren-13-ol, 8-hydroxy para-cymene, 8, 8-diethoxy-2,6-dimethyl-2-octanol, 8-para-menthen-2-ol, 9-decen-1-ol, 9-decen-1-yl acetate, α-amyl cinnamaldehyde dimethyl acetal, α-angelica lactone, α-bisabolol, α-cedrene epoxide, α-furaldehyde, α-furfuryl pentanoate, α-hexyl cinnamaldehyde, α-irone, α-methyl cinnamaldehyde, α-methyl naphthyl ketone, α-methyl-α-ethyl acrolein, α-phellandrene, α-phenyl ethyl acetate, α-pinene, α terpinene, α-terpinene, α-terpineol, α-tolyl aldehyde dimethyl acetal, α,β-fenchyl acetate, α-angelica lactone, α-cedrene epoxide, α-damascone, α-iso-methyl ionone, α-n-amyl-β-phenyl acryl acetate, α-phellandrene, ambroxan, amyl cinnamaldehyde dimethyl acetal, β-caryophyllene, β-damascenone, β-damascone, β-ionone, β-naphthyl ethyl ether, β-naphthyl methyl ether, β-naphthyl methyl ketone, β-pinene, β-caryophyllene, β-homo cyclocitral, β-homocyclocitral, β-ionone, carvyl acetate, carvyl propionate, cedryl methyl ether, cis-2,6-cis-21,22-α-irone, citral, citral diethyl acetal, citral dimethyl acetal, citronellol, damascenone, delta-amyl-delta-valerolactone, delta-damascone, delta-decalactone, delta-nonalactone, delta-octalactone, delta-propyl-delta-valerolactone, delta-tetradecalactone, delta-undecalactone, delta-damascone, dextra-carvone, dextro-carvone, dextro-fenchone, dextro-limonene, dextro-(+)-camphor, dextro-2-camphanone, dextro-fenchone, dextro-limonene, dihydrocoumarin, dihydroeugenol, dihydroeugenol, dimethyl benzyl carbinol, dimethyl benzyl carbinyl formate, dimethyl octanol, farnesene, fenchyl acetate, γ-bisabolene, γ-butyly-butyrolactone, γ-butyrolactone, γ-decalactone, γ-ethyl-γ-butyrolactone, γ-heptalactone, γ-hexalactone, γ-hexyl-γ-butyrolactone, γ-methyly-butyrolactone, γ-nonalactone, γ-octalactone, γ-phenyl allyl acetate, γpropyl-γ-butyrolactone, γ-terpinene, γ-undecalactone, γ-valerolactone, γ-bisabolene, γ-terpinene, geosmin, geranic oxide, geraniol, geranyl acetate, geranyl acetone, geranyl butyrate, geranyl formate, geranyl isobutyrate, geranyl propionate, homo furonol, hydroxycitronellal, hydroxycitronellal dimethyl acetal, hydroxycitronellol, iso amyl 2-methyl butyrate, iso amyl acetate, iso amyl benzoate, iso amyl butyrate, iso amyl cinnamate, iso amyl formate, iso amyl hexanoate, iso amyl isobutyrate, iso amyl isovalerate, iso amyl laurate, iso amyl octanoate, iso amyl ortho-hydroxybenzoate, iso amyl phenyl acetate, iso amyl propionate, iso amyl salicylate, iso bornyl acetate, iso bornyl alcohol, iso bornyl propionate, iso butyl acetate, iso butyl benzoate, iso butyl butyrate, iso butyl caproate, iso butyl cinnamate, iso butyl crotonate, iso butyl formate, iso butyl hexanoate, iso butyl isobutyrate, iso butyl methyl ketone, iso butyl ortho-hydroxybenzoate, iso butyl phenyl acetate, iso butyl propionate, iso butyl salicylate, iso butyl trans-2-butenoate, iso butyl valerate, iso dihydrolavandulal, iso eugenol, iso eugenyl acetate, iso eugenyl benzyl ether, iso eugenyl methyl ether, iso eugenyl phenyl acetate, iso hexenyl tetraydrobenzaldehyde, iso longifolanone, iso menthone, iso methyl-α-ionone, iso pentyl 2-methyl butanoate, iso pentyl 3-phenyl propenoate, iso pentyl benzoate, iso pentyl butyrate, iso pentyl dodecanoate, iso pentyl hexanoate, iso pentyl isobutyrate, iso pentyl isopentanoate, iso pentyl phenyl acetate, isopropyl acetate, isopropyl α-methyl crotonate, isopropyl butyrate, isopropyl cinnamate, isopropyl tiglate, iso pulegol acetate, isobornyl propionate, isodihydrolavandulal, isolongifolanone, isopulegyl acetate, ketoiso phorone, laevo-borneol, laevo-bornyl acetate, laevo-carveol, laevo-carvone, laevo-limonene, laevo-menthol, laevo-menthyl acetate, laevo-perillaldehyde, laevo-rose oxide, laevo-4-iso propenyl-1-cyclohexene-1-carboxildehyde, laevo-borneol, laevo-carveol, laevo-limonene, laevo-menthyl acetate, leerall, linalool, linalool oxide, linalyl acetate, linalyl propionate, longifolene, maltol, menthalactone, methyl cyclopentenolone, methyl ionone, methyl ionone, methyl-ionone, musk gx, musk ketone, myrtenal, myrtenol, myrtenyl acetate, nerol, nerol oxide, neryl isobutyrate, nootkatone, nopol, nopy acetate, ocimen quintoxide, oleic acid, omega-pentadecalactone, omega-6-hexadecenlactone, ortho-aminomethyl benzoate, ortho-methoxycinnamaldehyde, ortho-methoxyphenyl acetate, ortho tert-butyl cyclohexyl acetate, ortho-tert-butyl cyclohexanone, ortho-tert-butyl cyclohexyl acetate, para-acetyl anisole, para-cresol, para-cresyl acetate, para-cresyl caprylate, para-cresyl methyl ether, para-cresyl phenyl acetate, para-cymene, para-hydroxybenzyl acetone, para-methoxybenzaldehyde, para-methoxybenzyl acetate, para-methyl anisole, para-methyl phenol, para-methyl phenyl phenyl acetate, para-propenyl phenyl methyl ether, para toluquinoline, para tolyl acetate, para-tolyl aldehyde, para, para, α-trimethyl benzyl alcohol, para-cresyl caprylate, para-cymene, para-isopropyl benzaldehyde, para-isopropyl-α-methyl hydrocinnamaldehyde, para-tert-butyl cyclohexyl acetate, para-tert-butyl-methyl dihydrocinnamaldehyde, phenethyl pivilate, safranal, salicylaldehyde, santalol, santalol, sclareolide, sec-butyl 3-methyl butane thioate, sec-butyl thioisovalerate, syringa aldehyde, terpinolene, terpinyl acetate, terpinyl propionate, tetrahydrolinalool, tetrahydromyrcenol, theaspirane, thymol, trans-anethol, trans-3-phenyl-2-propene nitrile, trans-α-ionone, trans-β-ocimene, trans-β-ocimene, tricyclodecyl acetate, trimethyl cyclopentenyl acetaldehyde, undecyl alcohol, undecylenic alcohol, valencene, verbenol and vetiverol.

In a preferred embodiment, a composition of the present invention comprises nine components, the nine components being benzyl benzoate, an aldehyde component, amyl salicylate, frankincense, extract of roses, extract of Cananga, extract of Piper, extract of Bursera and at least one auxiliary component selected from the group consisting of vanillin, ethyl vanillin, extract of Hedychium, extract of Menta, extract of Citrus and mixtures thereof.

Preferably the benzyl benzoate comprises between about 0.1% and about 60% by weight of the total weight of the nine components in the composition.

Preferably the aldehyde component comprises between about 0.1% and about 55% by weight of the total weight of the nine components in the composition. The aldehyde component is preferably a mixture of the alkyl aldehydes described hereinabove.

Preferably the amyl salicylate is added in an amount so as to comprise between about 0.1% and about 55% by weight of the total weight of the nine components in the composition. The amyl salicylate is iso-amyl salicylate, n-amyl salicylate or a mixture thereof.

Preferably the frankincense is added in an amount so as to comprise between about 0.1% and about 40% by weight of the total weight of the nine components in the composition. Most preferably, the source of the frankincense is Boswellia carteri.

Preferably the extract of roses is added in an amount so as to comprise between about 0.1% and about 40% by weight of the total weight of the nine components in the composition. Preferably, the extract of roses is an extract of rose flowers, preferably of rose petals, preferably rose petals of Rosa damascena, especially Rosa damascena grown in Bulgaria. Preferably the extract of rose petals is rose oil, rose oil absolute or a mixture thereof.

Preferably, the extract of Cananga is added in an amount so as to comprise between about 0.1% and about 40% by weight of the total weight of the nine components in the composition. Preferably the extract of Cananga odorata is an extract of the flower of Cananga, especially the essential oil of the flower of Cananga, especially ylang ylang.

Preferably the extract of Piper comprises between about 0.1% and about 60% by weight of the total weight of the nine components in the composition. Preferably the extract of Piper comprises an extract of leaves of a plant, or comprises an extract of Piper auritum preferably an extract of leaves of Piper auritum. Preferably, the extract of Piper comprises an essential oil of leaves of Piper auritum.

Preferably the extract of Bursera comprises between about 0.1% and about 22% by weight of the total weight of the nine components in the composition. Preferably, the extract of Bursera comprises an extract of Bursera wood, preferably the extract of Bursera comprising linaloe wood oil. Preferably the extract of Bursera wood comprises oil extracted from Bursera glabrifolia and/or Bursera delpechiana.

Preferably the auxiliary component comprises up to about 60% by weight of the composition.

In an embodiment of the preferred composition of the present invention, the auxiliary component comprises vanillin and at least one additional auxiliary component selected from the group consisting of ethyl vanillin, extract of Hedychium, extract of Menta, extract of Citrus. In such an embodiment, the vanillin preferably comprises up to about 58% by weight of the composition.

In an embodiment of the preferred composition of the present invention, the auxiliary component comprises ethyl vanillin and at least one additional auxiliary component selected from the group consisting of vanillin, extract of Hedychium, extract of Menta, extract of Citrus. In such an embodiment, the ethyl vanillin preferably comprises up to about 58% by weight of the composition.

Especially preferred compositions of the present invention are described in the experimental section, hereinbelow.

The composition of the present invention is generally prepared by mixing or combining the separate components. Specifically, a composition such as the preferred embodiment described hereinabove is prepared by mixing benzyl benzoate, the aldehyde component, amyl salicylate, ethyl vanillin, frankincense and extracts of roses, extracts of Cananga, extracts of Piper, and extract of Bursera together. It is often preferable to first dissolve the frankincense in benzyl benzoate before adding the other components.

Generally a composition of the present invention is an oily substance that is very concentrated and uneconomical for use. It is thus generally preferable to provide an article of manufacture that includes a composition of the present invention together with a carrier. It is advantageous that such an article of manufacture be packaged in a packaging material and identified in print, in or on the packaging material, for use for overcoming effects of malodor.

Articles of manufacture that advantageously can comprise the composition of the present invention include, but are not limited to fabric-care products, personal hygiene products, air-freshening products, cleaning products and cosmetic products.

Suitable fabric-care products include but are not limited to laundry detergents, laundry soaps, fabric softeners, fabric sprays, fabric deodorants, dryer-added products, whitener products, bleaching products, optical whitener products and odor masking products.

Suitable cleaning products include but are not limited to bleach, cleaners, dishwashing products, toilet cleaning products and floor cleaning products.

Suitable personal hygiene products include but are not limited to shaving creams, shaving lotions, after shave lotions, soaps, shampoos, hair conditioners, deodorants, sun-screen products, bath salts and bath oils.

Suitable cosmetic products include but are not limited to perfumes, colognes, blushes, creams, face powders, lip balms and lipsticks.

An article of manufacture of the present invention can be provided with a solid carrier, for example such as sublimating air freshener gels, talc-based powder, carbon-based powder, fabric, cloth, tissue, paper, pledget, pad, nasal tampon, mask and dissolvable bath salts.

An article of manufacture of the present invention can be provided with a liquid carrier, for example such liquids as solutions, tinctures, oils, colognes, perfumes, eaux de parfum and eaux de toilette.

An article of manufacture of the present invention can be provided with a viscous fluid carrier for example a carrier such as balm, colloid, cream, emulsion, foam, gel, lotion, paste, sol, smearable stick suspension, and unguent. In a preferred embodiment of the present invention the article of manufacture is provided in a carrier such as petrolatum, fractionated coconut oil and bees' wax so as to produce a smearable viscous fluid. Typically in such an article of manufacture the carrier comprises at least about 85% by weight, generally between about 85% and about 99.9% by weight or between about 85% and about 97% by weight of the article. Typically in such an article of manufacture the carrier comprises no more than about 15% by weight, generally between about 0.1% and about 15% by weight or between about 0.5% and about 10% by weight of the article.

A method for reducing the negative effects of malodor on an individual according to the present invention is by positioning a composition of the present invention so that vapors emanating from the composition affect olfactory receptors of the individual thereby reducing the negative effects on the individual of malodor emerging from a source of malodors at a location.

Individuals that can advantageously make use of the teachings of the present invention include but are not limited to pregnant women, wounded persons, persons afflicted with cancer, persons afflicted with AIDS, persons undergoing medical treatment, persons afflicted with a foul smelling wounds, diabetics, health-care workers and rescue workers.

Sources of malodor, some of which are ameliorated by various embodiments of the present invention, include agriculture, amines, body odor, cigarette smoke, compost, diapers, dairy industry, gangrenous wounds, garbage, human feet, dirty laundry, halitosis, bad breath, lesions, livestock, manure, mercaptans, sewage, sludge, smoke, swine, tobacco, trash compactors, tumors, smoke, stale sweat, ulcers, unwashed humans, vomit, waste water, rotting proteins (flesh), decomposing proteins (flesh), burned proteins (flesh), abscesses, urine, viscera, offal, feces, ammonia, amines produced during the putrefaction of proteins (flesh) and indoles produced during the putrefaction of proteins (flesh).

Locations where such malodors are found include but are not limited to clinics, dental clinics, hospitals, surgical wards, mass graves, morgues, battlefields, abattoirs, industrial plants, sewers, earthquake loci, collapsed buildings, sewage processing plants, tanning plants, animal handling areas, barns, cancer wards, changing rooms, clarifiers, coal mines, composting sites, crematoria, crummy motels, diaper pails, dormitories, feed lots, garbage dumps, garbage processing plants, kennels, landfills, laundry rooms, leather processing plants, locker rooms, lumber mills, meat processing plants, milking parlors, mines, mothers in law, nursing homes, old age homes, outhouses, paper mills, photographic products manufacturing plants, poultry processing plants, prisons, rendering plants, settling basins, sewage dewatering systems, sludge stations and sport centra.

For use in reducing the negative effects of malodor associated with death, rotting and putrefying flesh and the like as found in locations including but not limited to hospitals, surgical wards, mass graves, morgues, battlefields, abattoirs, earthquake loci, collapsed buildings and the like, it is preferred to use a composition of the present invention that includes vanillin, ethyl vanillin or a combination thereof.

For use in reducing the negative effects of smells associated with cancer and cancer wards it is preferred to use a composition of the present invention that includes extract of Hedychium, especially Hedychium spicatum. Such a composition is exceptionally suited for use by such individuals as persons afflicted with cancer, persons undergoing medical treatment and health-care workers. Such a composition is exceptionally useful in neutralizing or reducing ancipatory nausea (apparently triggered by the smell associated with locations such as hospitals, surgical wards, cancer wards, nursing homes and old age homes) of patients undergoing chemotherapeutic treatment.

For use in reducing the negative effects of malodors that irritate or otherwise cause undesired effects in pregnant women it is preferred to use a composition of the present invention that includes extract of Menta, especially of Menta piperata.

For use in reducing the negative effects of malodors that are associated with fire, and smoke such as cigarette smoke, tobacco, burned oil or protein (flesh), charred protein (flesh) and ash but also smoke and the characteristic smells associated with fire it is preferred to use a composition of the present invention that includes extract of Citrus, especially of Citrus limonum.

A method of positioning the composition of the present invention that is both effective and economical involves using an article of manufacture of the present invention that is a smearable composition, such as the article that is substantially a composition of the present invention in a fluid or viscous fluid carrier, described above and in the Examples. The smearable composition is smeared in the vicinity of an olfactory organ of an organism, for example on the filtrum of a human. With every breath through the nose, an effective amount of perception altering vapors from the composition is inhaled irrespective of the positioning of the organism relative to the source of stench and malodor.

The method of altering smell perception using a composition of the present invention is also useful for treating an object contaminated with a malodor. According to the method of the present invention, the contaminated object is brought in contact with an appropriate composition-containing article as discussed hereinabove so that the object absorbs or adsorbs some of the composition and thus the negative effects associated with the malodor are reduced. For example, for contaminated hands or other body parts, a composition-containing soap is used. For contaminated clothing or other cloth/fabrics, a composition-containing laundry detergent is used. For contaminated air, for example emerging from air ducts, a sublimating composition-containing air freshener is used. For contaminated air, for example emerging from the mouth of a person, a composition-containing mouthwash, toothpaste or other dentifrice is used.

The method of altering smell perception using a composition of the present invention is also useful when the composition is positioned in the vicinity of the source of the malodor or placed at the location where the source of malodor is found. For example, air-freshening products, decorative items (such as artificial flowers or fountains), sublimating air fresheners, fabrics, cloths, tissues, sponges, papers, and pads including a composition are placed in the vicinity of the source of malodor or in a space where malodor is found.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following example, which together with the above description illustrates the invention in a non-limiting fashion.

### Materials and Experimental Methods

Aldehydes, benzyl benzoate, ethyl vanillin, vanillin are all commercially available and were purchased from Sigma-Aldrich (St. Louis, Mo., USA).

An equimolar mixture of aldehydes aldehyde C8 (CAS nr. 124-13-0), aldehyde C9 (CAS nr. 124-19-6), aldehyde C10 (CAS nr. 112-31-2), aldehyde C11 (CAS nr. 112-44-7), aldehyde C12 (CAS nr. 112-54-9), aldehyde C13/C15 (CAS nr. 9382-14-3), aldehyde C14 (CAS nr. 124-25-4) and aldehyde C16 (CAS nr. 77-83-8) was prepared.

Frankincense (from Boswellia carteri), rose oil (from Rosa damascena of Bulgarian origin), rose oil absolute (from Rosa damascena of Bulgarian origin), ylang ylang oil (from Cananga odorata), Mexican pepper leaf oil (from Piper auritum of Texan origin), essential oil of ginger lilly root (Hedychium spicatum), essential oil of peppermint (Menta piperata), essential oil of lemon (Citrus limonum) and linaloe wood oil (from Bursera glabriflora) are all commercially available and were purchased from Gritman Oils (Friendswood, Tex., USA).

Amyl salicylate, consisting of between 27% and 40% of the iso isomer together with the normal isomer (as determined by UV-GC) is commercially available and was purchased from International Flavors and Fragrances, Inc. (New York, N.Y., USA).

Toxicity studies of the first composition were conducted in accordance with EPA Pesticide Assessment Guidelines, Subdivision F, Hazard Evaluation: Human and Domestic Animals, Series 81-1 and OPPTS 870.1100 (for acute and oral toxicity potential), Series 81-2 and OPPTS NO. 870.1200 (for acute dermal toxicity potential), Series 81-4 and OPPTS N. 870.2400 (for eye irritation potential), and Series 81-5 and OPPTS No. 870.2500 (for dermal irritation potential). In addition, sensitizing potential was determined using a modification of the Buehler method (Rithz, H. L. and Buehler, E. V., “Planning, Conduct, and Interpretation of Guinea Pig Sensitization Patch Tests,” Concepts in Cutaneous Toxicity, p. 28, Academic Press, N.T., 1980) in accordance with EPA Pesticide Assessment Guidelines, Subdivision F, Hazard Evaluation: Human and Domestic Animals, Series 81-6 and OPPTS No. 870.2600.

### Experimental Results

Preparation of a First Composition of the Present Invention

0.2 g of frankincense were dissolved in 0.5 g of benzyl benzoate. After complete dissolution of the frankincense, 0.4 g of the aldehyde mixture, 0.4 g of amyl salicylate, 0.8 g of ethyl vanillin, 0.1 g of vanillin, 0.2 g of rose oil, 0.2 g of rose oil absolute, 0.2 g of ylang ylang oil, 0.5 g of Mexican pepperleaf oil and 0.1 g of linaloe wood oil were added and mixed together to yield a first composition of the present invention.

A smearable product of the present invention in a carrier was prepared by adding 1 gram of the first composition described above with 9 g of pharmaceutical grade white petrolatum.

Preparation of a Second Composition of the Present Invention

0.2 g of frankincense were dissolved in 0.5 g of benzyl benzoate. After complete dissolution of the frankincense, 0.4 g of the aldehyde mixture, 0.4 g of amyl salicylate, 0.8 g of essential oil of ginger lilly root (Hedychium spicatum), 0.1 g of vanillin, 0.2 g of rose oil, 0.2 g of rose oil absolute, 0.2 g of ylang ylang oil, 0.5 g of Mexican pepperleaf oil and 0.1 g of linaloe wood oil were added and mixed together to yield a second composition of the present invention.

A smearable product of the present invention in a carrier was prepared by adding 1 gram of the second composition described above with 9 g of pharmaceutical grade white petrolatum.

Preparation of a Third Composition of the Present Invention

0.2 g of frankincense were dissolved in 0.5 g of benzyl benzoate. After complete dissolution of the frankincense, 0.4 g of the aldehyde mixture, 0.4 g of amyl salicylate, 0.8 g of essential oil of peppermint (Menta piperata), 0.1 g of vanillin, 0.2 g of rose oil, 0.2 g of rose oil absolute, 0.2 g of ylang ylang oil, 0.5 g of Mexican pepperleaf oil and 0.1 g of linaloe wood oil were added and mixed together to yield a third composition of the present invention.

A smearable product of the present invention in a carrier was prepared by adding 1 gram of the third composition described above with 9 g of pharmaceutical grade white petrolatum.

Preparation of a Fourth Composition of the Present Invention

0.2 g of frankincense were dissolved in 0.5 g of benzyl benzoate. After complete dissolution of the frankincense, 0.4 g of the aldehyde mixture, 0.4 g of amyl salicylate, 0.8 g of essential oil of lemon (Citrus limonum), 0.1 g of vanillin, 0.2 g of rose oil, 0.2 g of rose oil absolute, 0.2 g of ylang ylang oil, 0.5 g of Mexican pepperleaf oil and 0.1 g of linaloe wood oil were added and mixed together to yield a fourth composition of the present invention.

A smearable product of the present invention in a carrier was prepared by adding 1 gram of the fourth composition described above with 9 g of pharmaceutical grade white petrolatum.

Efficacy of the First Product in Eliminating the Offensive Perception of Malodor and Stench

A small amount of the first smearable product of the present invention was smeared on the filtrum of a blindfolded human subject.

Pure and mixtures of putrescine, cadaverene and skatole were placed in test tubes. The human subject was allowed to sniff the odors emanating from the test tubes. The subject did not report any of the usual reactions to such odors (headache, nausea, diarrhea, hoarseness, chest tightness, nasal congestion, shortness of breath, stress, drowsiness or mood alteration.

In three separate flasks were placed 50 g of feces, 50 g of putrefied cow flesh and 50 g of urine respectively. The human subject was allowed to sniff the odors emanating from the mouths of the three flasks. In none of the cases was discomfort reported by the subject.

60 kg of decaying flesh and chocolate chip cookies were placed in a sealed room for a period of 1 week. The human subject entered the room and reported being able to smell the chocolate chip cookies. Further, the subject did not report any of the usual reactions to the smell of decaying flesh.

A first test mixture of 1 mg skatole and 1 mg cadaverine in 1 gram petroleum jelly was made so that there was an approximately 1:1 perceptual balance of the two components. The first mixture was succeedingly diluted by 50% to make an additional seven mixtures having concentrations, relative to the first mixture of ½, ¼, ⅛, 1/16, 1/32, 1/64 and 1/128. The samples were placed in a vapor delivery device provided with eight pairs of two air-emitting cones. One cone of each pair delivered clean air while the other cone delivered air contaminated with vapors from one of the eight samples. For testing, 40 liter min⁻¹ air was emitted from each cone with a linear speed of 8 cm sec⁻¹. The vapor concentration of cadaverine was measured at the cone, from most to least concentrated, to be 3.2, 1.6, 0.8, 0.4, 0.2, 0.09, 0.05 and 0.02 ppb.

Ten young healthy adult humans (five males and five females) were chosen as test subjects. Each subject participated in two days of testing within one week. On the first day of each subject, the subject performed four separate testing sessions of 99 minutes each session. During each session, a subject was exposed nine times to each of the eight test mixtures. Between the second and third session there was a lunch break.

On the first day of testing, during the first and fourth sessions each subject had white petroleum jelly applied on the septum. During the second and third sessions each subject had a sample of the first smearable product of the present invention applied on the septum. On the second day of testing, during the second and third sessions each subject had white petroleum jelly applied on the septum. During the first and fourth sessions each subject had a sample of the first smearable product of the present invention applied on the septum.

During each trial a subject was presented with the pairs of air-emitting cones, from lowest to highest concentration, and asked to choose the cone from the two with the strongest odor and to rate the confidence in this choice on a scale of 1 (uncertain) to 5 (very certain). After comparing all eight pairs a subject was given at least five minutes rest.

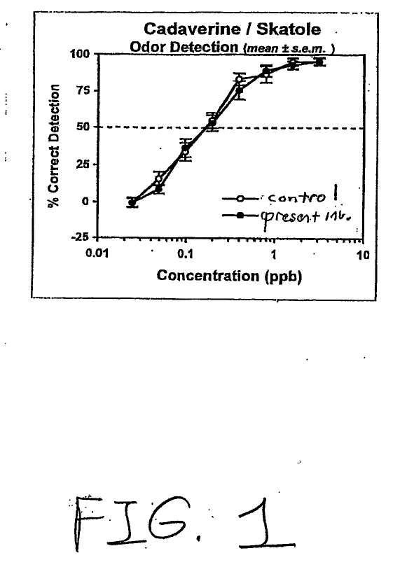

In FIG. 1 is shown a graph of odor detection as a function of cadaverine concentration. Filled circles are results with a smearable product of the present invention while empty circles are results with petroleum jelly. From the graph in FIG. 1 it is seen that the ability to sense the odors of cadaverine and skatole was not significantly affected by the use of a product of the present invention.

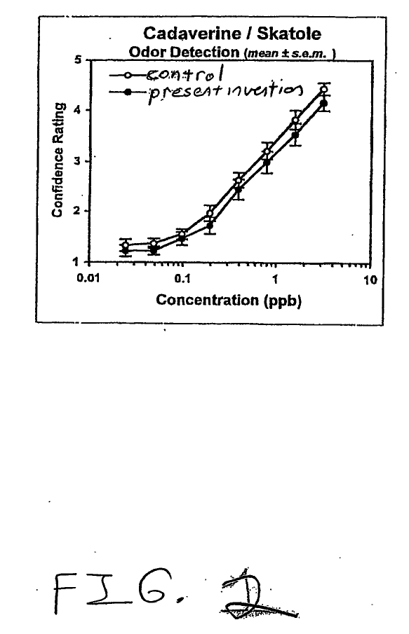

In FIG. 2 is shown a graph of the confidence rating. It is clearly seen that use of a product of the present invention has a significant effect on the confidence of detection of an offensive smell.

The results show that use of the first smearable product of the present invention reduces confidence in the perceived presence of skatole and cadaverine to a statistically significant degree. This is understood to mean that the use of the composition of the present invention allows detection of the mixtures but neutralizes the exceptional offense usually sensed upon exposure to such a mixture.

Acute Eye Irritation Study in Rabbits

Based on the Maximum Average Irritation Score of 4.0, the first smearable product of the present invention was rated minimally irritating. As no “positive” effects were observed, the first composition was assigned to Toxicity Category IV. No irritation was observed in any eye after 24 hours.

Acute Oral Toxicity Study in Rats

Acute oral LD₅₀, of the first composition was found to be greater than 5050 mg/kg in both males and females. The test composition was assigned to Toxicity Category IV.

Acute Dermal Irritation Study in Rabbits

Erythema, edema nor other signs of irritation-were observed. The primary index of 0.0 from a of maximum 8.0 was obtained after 1, 24, 48 and 72 hours, giving the first composition a descriptive rating of non-irritating. Based on the 72-hour observations the Toxicity Category is IV.

No skin sensitization was observed.

Efficacy of the First Product in Improving Functioning Under Difficult Conditions

Fifty rescue workers are sent to the site of an earthquake where many decaying corpses are trapped under collapsed building. A small amount of the first smearable product of the present invention is smeared on the filtrum of a first group of twenty-five of the rescue workers. A small amount of white petroleum jelly spiked with vanillin and ethyl vanillin in amounts equivalent to that found in the first smearable product is smeared on the filtrum of a second group of twenty-five of the rescue workers. The workers of both groups are advised to replenish the applied smearable product when desired. Over a period of five days the number of work hours compared to rest hours and sick hours as well as the volume of waste searched by each of the two groups is recorded. It is seen that the first group performed the search work more efficiently and with less lost hours than the second group.

Efficacy of the Second Product in Eliminating Anticipatory Nausea in Cancer Patients

Before arriving at a hospital for chemotherapeutic treatment, a small amount of the second smearable product of the present invention is smeared on the filtrum of a first group of persons afflicted with cancer while a small amount of white petroleum jelly spiked with vanillin and ginger lilly root oil in amounts equivalent to that found in the second smearable product is smeared on the filtrum of a second group of persons afflicted with cancer.

The first group reports no or significantly reduced levels of anticipatory nausea caused by smelling the hospital and specifically, detecting the characteristic smells of a chemotherapy clinic.

Efficacy of the Third Product in Eliminating the Perception of Offensive Smells by Pregnant Women

A group of pregnant women are exposed to smells known to irritate pregnant women including frying bacon, burnt chicken feathers, baked sausages and household garbage. Women reporting that one or more such smells caused distress, nausea, headache or other discomfort were selected to be part of a test group.

To a first subgroup of the test group, a small amount of the third smearable product of the present invention is smeared on the filtrum while a small amount of white petroleum jelly spiked with vanillin and peppermint oil in amounts equivalent to that found in the third smearable product is smeared on the filtrum of a second subgroup of the test group.

The first subgroup reports no or significantly reduced levels of discomfort compared to the second subgroup.

Efficacy of the Fourth Product in Eliminating the Offensive Perception of Fire and Smoke

A group of people is exposed to smells associated with smoke and fire including cigarette smoke, pipe-smoke, old ashtrays, burnt plastic and blankets from the site of a burned house. People reporting that one or more such smells caused distress, nausea, headache or other discomfort were selected to be part of a test group.

To a first subgroup of the test group, a small amount of the fourth smearable product of the present invention is smeared on the filtrum while a small amount of white petroleum jelly spiked with vanillin and lemon oil in amounts equivalent to that found in the second smearable product is smeared on the filtrum of a second subgroup of the test group.

The first subgroup reports no or significantly reduced levels of discomfort compared to the second subgroup.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific examples thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A composition comprising at least three plant extracts wherein said composition is effective in altering the perception of at least one malodor by affecting at least one olfactory receptor.

2. The composition of claim 1, wherein said three plant extracts are selected from the group consisting of frankincense, extract of roses, extract of Cananga, extract of Piper, and extract of Bursera.

3. The composition of claim 2, comprising four plant extracts selected from the group consisting of frankincense, extract of roses, extract of Cananga, extract of Piper, and extract of Bursera.

4. The composition of claim 2, comprising five plant extracts: frankincense, extract of roses, extract of Cananga, extract of Piper, and extract of Bursera.

5. (canceled)

6. The composition of claim 2, wherein said frankincense comprises between about 0.1% and about 40% by weight of the composition.

7-12. (canceled)

13. The composition of claim 2, wherein said extract of roses comprises between about 0.1% and about 40% by weight of the composition.

14-17. (canceled)

18. The composition of claim 2, wherein said extract of Cananga comprises between about 0.1% and about 40% by weight of the composition.

19-22. (canceled)

23. The composition of claim 2, wherein said extract of Piper comprises between about 0.1% and about 60% by weight of the composition.

24-26. (canceled)

27. The composition of claim 2, wherein said extract of Bursera comprises between about 0.1% and about 22% by weight of the composition.

28. The composition of claim 2, further comprising at least one auxiliary component selected from the group consisting of vanillin, ethyl vanillin, extract of Hedychium, extract of Menta, extract of Citrus and mixtures thereof.

29. The composition of claim 28, wherein said auxiliary component comprises up to about 60% by weight of the composition.

30-42. (canceled)

43. The composition of claim 2, further comprising benzyl benzoate.

44. (canceled)

45. The composition of claim 2, further comprising an aldehyde component.

46. The composition of claim 45, wherein said aldehyde component comprises a mixture of one or more hydrocarbon aldehydes of a structure RCH═O, wherein R is selected from the group consisting of aryl and alkyl groups.

47-48. (canceled)

49. The composition of claim 45, wherein said aldehyde component comprises between about 0.1% and about 55% of the composition.

50. The composition of claim 2, further comprising amyl salicylate.

51-52. (canceled)

53. The composition of claim 2, wherein said amyl salicylate comprises between about 0.1% and about 55% by weight of the composition.

54-58. (canceled)

59. A composition comprising frankincense, extract of roses, as, extract of Cananga, extract of Piper and extract of Bursera.

60-67. (canceled)

68. The composition of claim 59, wherein said frankincense comprises between about 0.1% and about 40% by weight of the composition.

69-74. (canceled)

75. The composition of claim 59, wherein said extract of roses comprises between about 0.1% and about 40% of the composition.

76-79. (canceled)

80. The composition of claim 59, wherein said extract of Cananga comprises between about 0.1% and about 40% by weight of the composition.

81-83. (canceled)

84. The composition of claim 59, wherein said extract of Piper comprises between about 0.1% and about 60% by weight of the composition.

85-87. (canceled)

88. The composition of claim 59, wherein said extract of Bursera comprises between about 0.1% and about 22% by weight of the composition.

89-102. (canceled)

103. An article of manufacture comprising the composition of claim 1 and a carrier.

104. (canceled)

105. The article of manufacture of claim 103, wherein said carrier is selected from the group of fabric-care products, personal hygiene products, air-freshening products, cleaning products and cosmetic products.

106-113. (canceled)

114. The article of manufacture of claim 103, wherein said carrier is a liquid.

115. (canceled)

116. The article of manufacture of claim 103, wherein said carrier is a viscous fluid.

117-125. (canceled)

126. The article of manufacture of claim 103, wherein said carrier is a solid.

127-131. (canceled)

132. A method of reducing the negative effects of malodor on an individual comprising positioning a composition of claim 1 so that vapors emanating from said composition affect olfactory receptors of the individual thereby reducing the negative effects on the individual of malodor emerging from a source of malodors at a location.

133. (canceled)

134. The method of claim 132, wherein said source is selected from the group of agriculture, amines, body odor, cigarette smoke, compost, dairy industry, gangrenous wounds, garbage, human feet, diapers, dirty laundry, halitosis, bad breath, lesions, livestock, manure, mercaptans, sewage, sludge, smoke, swine, tobacco, trash compactors, tumors, smoke, stale sweat, ulcers, unwashed humans, vomit, waste water, rotting proteins, decomposing proteins, burned proteins, abscesses, urine, viscera, offal, feces, ammonia, amines produced during the putrefaction of proteins and indoles produced during the putrefaction of proteins and combinations thereof.

135. The method of claim 132, wherein said location is selected from the group consisting of clinics, dental clinics, hospitals, surgical wards, mass graves, morgues, battlefields, abattoirs, industrial plants, sewers, earthquake loci, collapsed buildings, sewage processing plants, tanning plants, animal handling areas, barns, cancer wards, changing rooms, clarifiers, coal mines, composting sites, crematoria, diaper pails, dormitories, feed lots, garbage dumps, garbage processing plants, kennels, landfills, laundry rooms, leather processing plants, locker rooms, lumber mill, meat processing plants, milking parlors, mines, nursing homes, old age homes, outhouses, paper mills, photographic products manufacturing plants, poultry processing plants, prisons, rendering plants, settling basins, sewage dewatering systems, sludge stations and sport centra and combinations thereof.

136. The method of claim 132, wherein said composition comprises an auxiliary component selected from the group consisting of vanillin, ethyl vanillin or a combination thereof.

137. The method of claim 136, wherein said source is found in a location selected from the group consisting of hospitals, surgical wards, mass graves, morgues, battlefields, abattoirs, earthquake loci, collapsed buildings, and combinations thereof.

138. The method of claim 132, wherein said composition comprises extract of Hedychium.

139. (canceled)

140. The method of claim 138, wherein said source is found in a location selected from the group consisting of hospitals, surgical wards, cancer wards, nursing homes, old age homes and combinations thereof.

141. The method of claim 132, wherein said composition comprises extract of Menta.

142. The method of claim 141, wherein said individual is a pregnant woman.

143. The method of claim 132, wherein said composition comprises extract of Citrus.

144. The method of claim 143, wherein said source includes a source selected from the group of fire, cigarette smoke, tobacco, burned oil or protein (flesh), charred protein (flesh) and smoke.

145. (canceled)

146. The method of claim 132, wherein said positioning comprises positioning said composition in the vicinity of said source or placing said composition at said location.

147-148. (canceled)

149. The method of claim 132, wherein said positioning comprises of the individual.

150-152. (canceled)

153. The method of claim 132, wherein said positioning comprises contacting said source of malodor with said composition.

154. (canceled)

155. The composition of claim 59, further comprising at least one auxiliary component selected from the group consisting of vanillin, ethyl vanillin, extract of Hedychium, extract of Menta, extract of Citrus and mixtures thereof.

156. The composition of claim 59, further comprising benzyl benzoate.

157. The composition of claim 59, further comprising an aldehyde component.

158. The composition of claim 59, further comprising amyl salicylate.

159. A composition useful in preparing a composition effective in altering the perception of at least one malodor by affecting at least one olfactory receptor comprising at least three different plant extracts, said three plant extracts selected from the group consisting of frankincense, extract of roses, extract of Cananga, extract of Piper, and extract of Bursera.
